(19) United States
(12) Patent Application Publication
(10) Pub. No.: US 2007/0224293 A1
(43) Pub. Date: Sep. 27, 2007

(54) Virus-interacting layered phyllosilicates and methods of inactivating viruses on animate and inanimate surfaces

(75) Inventors: John Hughes, University Park, FL (US); Jerald W. Darlington JR., Marengo, IL (US); Panayiotis P. Constantinides, Gurnee, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AMCOL INTERNATIONAL, Arlington Heights, IL (US)

(21) Appl. No.: 11/641,319

(22) Filed: Dec. 18, 2006

Related U.S. Application Data
(63) Continuation-in-part of application No. 11/196,090, filed on Aug. 3, 2005.

(51) Int. Cl. A61K 33/12; A61K 33/06

(52) U.S. Cl. 424/683; 424/684

(57) ABSTRACT

Layered phyllosilicates are useful for adsorbing and/or binding to and, thereby, inactivating viruses. Accordingly, provided herein is a method of inhibiting transfer of a virus to a surface comprising contacting the surface with a composition comprising a layered phyllosilicate material in an amount sufficient for inhibiting the transfer of the virus thereto. Also provided are methods of inactivating a virus on a surface comprising contacting the surface with a composition comprising a layered phyllosilicate material in an amount sufficient to inactivate said virus.

This application is a continuation-in-part of U.S. patent application Ser. No. 11/196,090, filed Aug. 3, 2005, the disclosure of which is incorporated herein by reference in its entirety.

### FIELD

Described herein are virucidal layered phyllosilicates capable of interacting with and thereby inactivating significant percentages of bacteria and a plurality of viruses.

### BACKGROUND

The number of people who were infected with HIV rose to its highest level ever in 2004. The WHO estimated a global total of 39.4 million people living with HIV and that 3.1 million people died of the infection in 2004 (www.unaids.org/wad2004/report.html). Of the world's HIV-infected individuals 50% with teenage girls accounting for 30% of the HIV infected women in some sub-Saharan African countries. Although contraception is available, the HIV epidemic continues to spread highlighting the urgent need for new prevention strategies (Balzarini, J. 2005). Virucides are of interest because they can act quickly and are more direct by binding to the virus coat proteins or viral membranes on contact (Al-Jabri, A. A et al., 2000). A number of HIV virucides are currently under investigation including the physical method of absorbing the virus using mineral clays, a method tried and tested by a number of scientists (Quignon, F. et al. 1997; Clark, K. J., Sarr, A. B., Grant, P. G., Phillips, T. D. & Woode, G. N., 1998; Meschke, J. S. & Sobsey, M. D., 2003). The adsorption effects of bentonite clay in the adsorption of viruses (Sobsey, M. D. and Cromeans, T., 1985; Lipson, S. M. & Stotzky, G., 1985), for example, have been studied extensively in the last few decades due to its use in microbial filtration in the treatment of water.

Further, in the past century we have witnessed three pandemics of influenza, of which the “Spanish flu” of 1918 was the largest pandemic of any infectious disease known to medical science (Oxford, J.S., 2000). The three strains which caused these pandemics belong to group A of the influenza viruses and, unlike the other two groups (B and C), this group infects a vast variety of animals (poultry, swine, horses, humans and other mammals).

Influenza A viruses continue to cause global problems, both economically and medically (Hayden, F. G. & Palese, P., 2000). The recent South East Asian outbreaks of avian influenza in 2003 and 2004 are ideal examples of this.

Much has been done to control and prevent another pandemic from occurring with many anti-influenza products (vaccines and treatments) currently on the market. The most recognized of these is TAMIFLUOR® (oseltamivir phosphate), a neuraminidase inhibitor, which functions by preventing spread of the virus within the human body.

Scientists have, in the recent years, been looking to develop new drugs following novel strategies of coping with Influenza. With the numbers of such projects on the rise researchers have been focusing on different Influenza target sites in which to develop new vaccines and treatments. Fiers, W. et al. (2004), for example, have reported the efficacy of an M2e vaccine, which targets the less variable M2 transmembrane protein of the influenza virus. Another example is the “OX40 treatment”, which reduces the excessive immune response that accompanies Influenza infections and which can increase the severity of symptoms (Hussell, T. et al. (2004).

Acute viral gastroenteritis is a very common illness which occurs in both epidemic and endemic forms. It affects all age groups worldwide and also includes some of the commonly encountered traveler's diarrhea. This syndrome is recognized as being second in frequency only to the common cold among illnesses affecting U.S. families under epidemiological surveillance. The clinical presentation of the illness is variable, but in general it is self-limited, has an explosive onset, and is manifested by varying combinations of diarrhea, nausea, vomiting, low-grade fever, abdominal cramps, headache, anorexia, myalgia, and malaise. It is not only responsible for a great deal of misery and time lost from school and work, but can be severe, indeed fatal, in the infant, elderly, or debilitated patient. Due to associated malabsorption, viral gastroenteritis may trigger or enhance the morbidity associated with malnutrition in marginally nourished populations. See Cukor et al., (Microbiol. Rev., 48:157-179, 1984) for further review of viral gastroenteritis.

The recent outbreaks of foot-and-mouth disease (FMD) in a number of FMD-free countries, in particular Taiwan in 1997 and the United Kingdom in 2001, have significantly increased public awareness of this highly infectious disease of cloven-hoofed livestock. Outbreaks have occurred in every livestock-containing region of the world with the exception of New Zealand, and the disease is currently enzootic in all continents except Australia and North America. The disease affects domestic cloven-hoofed animals, including cattle, swine, sheep, and goats, as well as more than seventy species of wild animals, including deer (Fenner et al., Veterinary Virology, p. 403-430, 1993), and is characterized by fever, lameness, and vesicular lesions on the tongue, feet, snout, and teats. Other vesicular diseases, such as swine vesicular disease (SVD), vesicular stomatitis, and vesicular exanthema of swine, cause signs so similar to those of FMD that differential clinical diagnosis alone can be difficult (Bachrach et al., Annu. Rev. Microbiol. 22:201-244, 1968). Although FMD does not result in high mortality in adult animals, the disease has debilitating effects, including weight loss, decrease in milk production, and loss of draught power, resulting in a loss in productivity for a considerable time. Mortality, however, can be high in young animals, where the virus can affect the heart. In addition, cattle, sheep, and goats can become carriers, and cattle can harbor virus for up to 2 to 3 years (Brooksby et al., Intervirology, 18:1-23, 1982). See Grubman et al., (Clin. Microbiol. Rev., 17:465-493) for a further review of foot and mouth disease.

Rhinoviruses are the most significant microorganisms in causing the acute respiratory illness referred to both physicians and lay persons as the “common cold”. Other viruses, such as parainfluenza viruses, respiratory syncytial viruses, enteroviruses, and coronaviruses, are known to cause symptoms of the “common cold”; however, rhinoviruses are now thought to cause the greatest amount of cases of the common cold. Rhinoviruses have also been found to be among the most difficult to kill of the cold causing viruses. While the molecular biology of rhinoviruses is now understood in great detail, the progress in determining effective methods for preventing colds caused by rhinoviruses and for preventing the spread of the virus to non-infected subjects has been slow.

Gwaltney et al., Annals of Internal Medicine, 88:463-467 (1978) reports that rhinovirus colds are commonly spread amongst a human population by hand-to-hand transmission. That is, one subject with a rhinovirus cold will have a brief physical contact with another subject. By virtue of the physical contact, rhinovirus particles will be present on the hands of the contacted person. The contacted person will then become infected with the rhinovirus by placing contaminated fingers on their nasal and conjunctival mucosa. This hand contact/self inoculation concept for rhinovirus transmission is supported by other research groups that have demonstrated that rhinovirus is recoverable from the hands of a large number of patients infected with rhinovirus.

Hendley et al., Antimicrobial Agents and Chemotherapy, 14:690-694 (1978) explored the idea of interrupting the spread of rhinoviruses through the use of antimicrobial liquids and foams applied to the hands. While it is acknowledged that frequent hand washing with ordinary soap and water will mechanically remove viruses from the hands and, thus, reduce the spread of rhinovirus colds, washing one's hands multiple times is irritating to the skin. Therefore, an objective of Hendley et al. was to evaluate the virucidal activity of certain agents that were believed to be non-irritating to the skin after multiple applications so that the need for mechanical removal of the virus by frequent washing was avoided. In Hendley et al., liquids containing dilute iodine in ethyl alcohol or water, and foams containing ethyl alcohol, benzalkonium chloride (BAK), and hexachlorophene were evaluated. The most effective treatment antiviral compositions contained iodine. In addition to immediately killing rhinoviruses on contact, iodine solutions were found to have a residual killing capacity that would inactivate rhinoviruses introduced on a subject's fingers for up to one hour after iodine application. By contrast, ethyl alcohol alone was not effective, and the combination of ethyl alcohol with BAK was fairly ineffective in killing rhinoviruses.

Despite the teachings in Hendley et al., iodine based washes are unsuitable for wide spread use in preventing the spread of rhinovirus induced colds. This is because iodine will cause some brown staining of a patient's hands and is somewhat irritating to the skin.

Hayden et al., Antimicrobial Agents and Chemotherapy, 26:928-929 (1984) also discussed the concept of interrupting the hand-to-hand transmission of rhinovirus colds through the use of a safe, cosmetically acceptable hand lotion which has lasting virucidal activity. In Hayden et al., it was discovered that hand lotions containing 2% glutaric acid were more effective than placebo in inactivating certain types of rhinovirus. However, Hayden et al. reports that the glutaric acid containing lotions were not effective against a full spectrum of rhinovirus serotypes.

In many settings, e.g., the family home, elementary school, in college dormitories, etc., people are in close contact for extended periods of time. As discussed above, this close contact is liable to result in an infected individual transmitting the virus to an unprotected individual. It would be advantageous to have a treatment regimen which can be used by both infected and non-infected individuals to halt the spread of the rhinovirus and other viruses.

Layered phyllosilicates, such as bentonite clay, or montmorillonite clay, are the active virus-interacting minerals described herein for inactivating viruses. Their virus sorption/binding properties, in prior art theory, are due to their negative electrical charge, which attracts positively charged toxins (including bacteria and viruses) and binds them. The virucidal phyllosilicates described herein, however, bind both positively charged and negatively charged virus molecules. It is theorized that sorption and/or binding of the virus to the layered phyllosilicates described herein is achieved by one or more mechanisms selected from the group consisting of adsorption; ionic complexing; electrostatic complexing; chelation; hydrogen bonding; ion-dipole; dipole/dipole; Van Der Waals forces; and any combination thereof. Such ionic bonding, e.g., via one or more cations or negative charge sites of the phyllosilicate sharing electrons with one or two atoms of one or two polar ends of a virus molecule, on a phyllosilicate surface, provides inactivation of a surprisingly high percentage of the virus molecules.

### SUMMARY IF THE INVENTION

It has been found that layered phyllosilicates are useful for adsorbing and/or binding to and, thereby, inactivating viruses. In one aspect, the viruses are the human immunodeficiency virus (HIV) and influenza A virus. The ability of a layered phyllosilicate to interact with and inactivate two very different acting viruses is most unexpected.

The layered phyllosilicate material useful for virus interaction, as described herein, includes the following clay minerals: montmorillonite, particularly sodium montmorillonite, protonated hydrogen montmorillonite, magnesium montmorillonite and/or calcium montmorillonite; nontronite; beidellite; laponite; yakhontovite; zincsilite; volkonskoite; hectorite; saponite; ferrosaponite; sauconite; swinefordite; pimelite; sobockite; stevensite; svinfordite; vermiculite; synthetic clays; mixed layered illite/smectite minerals, such as rectorite, tarosovite, and ledikite; admixtures of illites with the clay minerals named above, and the magnesium aluminum silicates. Any one or any mixture of two or more of the above clay minerals is capable of adsorbing, and/or ionically bonding with, any virus, or combination of viruses, thereby inactivating the virus(es).

One preferred layered phyllosilicate is a smectite clay having at least 80%, preferably at least 95% interlayer, exchangeable homolonic cations, preferably sodium ions, based on the total of number of interlayer, exchangeable cations. Other particularly effective phyllosilicates that are effective in interacting with and inactivating significant percentages of a host of viruses, particularly HIV and influenza A viruses, include protonated onium ion-exchanged layered phyllosilicates (protonated organoclays); smectite clays having a particle size less than 74 μm, preferably less than 50 μm, more preferably less than 20 μm; and exfoliated smectite clays, including individual clay platelets and tactoids of 5 or less platelet layers.

In accordance with one embodiment for using the virucidal layered phyllosilicates described herein, the phyllosilicate particles are sprayed onto an absorbent mask as an air purification device, or included in a hand wipe material (hand sanitizers) for cleaning virus-contaminated surfaces, thereby adsorbing and inactivating the viruses, thereby preventing viruses from being breathed into the nose and mouth of a person or for adsorbing and thereby inactivating viruses from the hands, e.g., before handling a baby; or on gloves to inactivate viruses.

In other embodiments, the virucidal layered phyllosilicates can be suspended in lotions or skin creams that are applied to skin, particularly hands and face, or internally within the vagina, for interacting with and thereby inactivating the transfer of viruses from one person to another, or to prevent a person from transferring the virus from external skin to internal cells.

In still another embodiment, the virucidal layered phyllosilicates can be ingested for internal interaction and inactivation of viruses within the gastrointestinal tract that have been or are about to be ingested. When wastes are expelled, viruses are retained on the clay and prevented from causing secondary infections.

In another embodiment, the virucidal layered phyllosilicates can be vaginally inserted for interaction and inactivation of HIV or other sexually-transmitted viruses, in the same manner as a spermicidal foam or body heat-dissolving spermicidal cartridge.

In still another embodiment, the virucidal layered phyllosilicates can be held in a vessel for filtering contact with blood, e.g., a secondary dialysis filter, or for filtering viruses from water in a virus-removing water purification step.

In another embodiment, the virucidal layered phyllosilicates can be used as, or form a portion of, a HVAC filtration media to prevent virus-contaminated air from passing between rooms, for example, between rooms in a hospital.

In another embodiment, the virucidal layered phyllosilicates are used as a nasal lubricant by spraying a suspension of the virucidal phyllosilicate in a carrier (water and/or organic solvent) into the nasal passages to coat nasal cells. In this manner, viruses entering the nose will interact with the phyllosilicate and thereby will be inactivated to prevent infection.

In still another embodiment, a condom is coated with a suspension of the virucidal layered phyllosilicates, in a cosmetically acceptable carrier, e.g., water and/or solvent. In the event of condom failure, the virucidal phyllosilicate interacts with and inactivates viruses before a sexual partner is infected.

In another embodiment, a suspension of the virucidal layered phyllosilicate in a cosmetically acceptable carrier is packaged in a portable container, e.g., a tube or bottle, for use on the hands to periodically inactivate viruses held on a person's skin.

In another embodiment, the virucidal layered phyllosilicates can be dispensed throughout a virus-contaminated body of water, such as a pond or lake, to inactivate viruses therein.

The virucidal layered phyllosilicates described herein interact with viruses, adsorb and/or bind them ionically to the virucidal layered phyllosilicates, thereby preventing the viruses from migrating to and penetrating cell membranes, thereby preventing the viruses from reproducing and rupturing the cells and releasing more of the virus.

In another embodiment, the invention provides a method of inhibiting transfer of a virus to a surface, the method comprising contacting the surface with a composition comprising a layered phyllosilicate material in an amount sufficient for inhibiting the transfer of the virus thereto. In some aspects, the composition is in a form selected from the group consisting of a solution, lotion, cream, ointment, powder, suspension, stick, gel, aerosol and nonaerosol pump spray. The surface can be an inanimate surface or an inanimate surface. In one aspect, the animate surface is on a mammalian subject selected from the group consisting of a human, a horse, a cow, sheep, a pig, a llama, an alpaca, a goat, a dog, a cat; a dromedary, an exotic animal, a zoo animal, a mouse, a rat, a rabbit, a guinea pig, and a hamster.

In one aspect, the layered phyllosilicate material comprises at least 90% homoionic interlayer exchangeable cations, in relation to all interlayer exchangeable cations, and has a particle size less than 74 μm. In another aspect, the phyllosilicate material comprises interlayer exchangeable cations that are predominantly hydrogen cations. In another aspect, the layered phyllosilicate material comprises exfoliated platelets and/or tactoids of the layered phyllosilicate material.

In another aspect, the virus is selected from the group consisting of Feline Calcivirus, Norovirus, Rotavirus, HSV-1, Influenza A, HIV and Rhinovirus.

In another embodiment, the invention provides a method of inactivating a virus on an animate surface comprising contacting said surface with a composition comprising a layered phyllosilicate material in an amount sufficient to inactivate said virus. Methods of inactivating a virus on an inanimate surface are also provided. In some aspects, the composition is in a form selected from the group consisting of a solution, lotion, cream, ointment, powder, suspension, stick, gel, aerosol and nonaerosol pump spray. In one aspect, the animate surface is on a mammalian subject selected from the group consisting of a human, a horse, a cow, sheep, a pig, a llama, an alpaca, a goat, a dog, a cat; a dromedary, an exotic animal, a zoo animal, a mouse, a rat, a rabbit, a guinea pig, and a hamster.

In one aspect, the layered phyllosilicate material comprises at least 90% homoionic interlayer exchangeable cations, in relation to all interlayer exchangeable cations, and has a particle size less than 74 μm. In another aspect, the phyllosilicate material comprises interlayer exchangeable cations that are predominantly hydrogen cations. In another aspect, the layered phyllosilicate material comprises exfoliated platelets and/or tactoids of the layered phyllosilicate material.

In another aspect, the virus is selected from the group consisting of Feline Calcivirus, Norovirus, Rotavirus, HSV-1, Influenza A, HIV and Rhinovirus.

### DETAILED DESCRIPTION OF THE INVENTION

Whenever used in this specification, the terms set forth shall have the following meanings:

Ranges may be expressed herein as from “about” or “approximately” one particular value and/or to “about” or “approximately” another particular value. When Such a range is expressed, another embodiment includes from the one particular value and/or to the other particular value. Similarly, when values are expressed as approximations, by use of the antecedent “about,” it will be understood that the particular value forms another embodiment.

As used herein, the terms “therapeutically effective” or “amount sufficient” refers to when a composition or method of the invention is properly administered ill vivo to a vertebrate, such as a bird or mammal, including humans, a measurable beneficial effect occurs. Exemplary beneficial effects include measurable antiviral effects in conditions where viral load can be assayed; a reduction of clinically verifiable and/or patient-reported symptoms or complete resolution or curing of the viral infection or outbreak.

As used herein, the term “antiviral activity” refers to the ability of the composition, method, or treatment regimen to reduce the size, extent, severity, and duration of infections, lesions, or the communicability of the virus.

As used herein, the tern “virucidal” means capable of inactivating or destroying a virus.

“Phyllosilicate” or “Virucidal Clay” shall mean clay minerals, e.g., montmorillonite, particularly sodium montmorillonite, magnesium montmorillonite and/or calcium montmorillonite; protonated montmorillonite; nontronite; beidellite; laponite; yakhontovite; zincsilite; volkonskoite; hectorite; saponite; ferrosaponite; sauconite; swinefordite; pimelite; sobockite; stevensite; svinfordite; vermiculite; synthetic clays; mixed layered illite/smectite minerals, such as rectorite, tarosovite, and ledikite; admixtures of illites with the clay minerals named above, and the magnesium aluminum silicates.

“Homoionic Phyllosilicate” shall mean a layered Phyllosilicate material that has been purified by ion-exchange, for example, as described in this assignee's U.S. Pat. No. 6,050,509, to contain at least 90% of a single element, in relation to all interlayer exchangeable cations, from periodic table groups 1a, 2a, 3b, 4b, 5b, 6b, 7b, 8, 1b, 2b, 3a, tin and lead; or a protonated onium ion compound, as the interlayer exchangeable cations.

“Platelets” shall mean individual layers of a Phyllosilicate.

“Intercalate” or “Intercalated” shall mean a phyllosilicate material that includes an onium ion spacing agent, preferably a protonated onium ion spacing agent, disposed between adjacent platelets of the layered Phyllosilicate material to increase the interlayer spacing between the adjacent platelets by at least 3 Å, preferably at least 5 Å, to an interlayer spacing, for example, of at least about 8 Å, preferably at least about 10 Å.

“Intercalation” shall mean a process for forming an Intercalate.

“Onium Ion Intercalant” or Onium Ion Spacing Agent” or “Onium Ion Compound” shall mean an organic compound, preferably a protonated organic compound, that includes at least one positively charged atom selected from the group consisting of a nitrogen atom, a phosphorous atom, a sulfur atom or an oxygen atom, preferably a quaternary ammonium compound, and when dissolved in water and/or an organic solvent, an anion dissociates from the onium ion spacing agent leaving an onium cation that can ion-exchange with a silicate platelet exchangeable cation of the Phyllosilicate, e.g., Na⁺, Ca⁺², Li⁺, Mg⁺², Al⁺³, or K⁺.

“Intercalating Carrier” shall mean a carrier comprising water and/or an organic liquid to form an Intercalating Composition capable of achieving Intercalation of an onium ion spacing agent which ion-exchanges with exchangeable interlayer cations of the layered Phyllosilicate.

“Intercalating Composition” shall mean a composition comprising one or more onium ion spacing agents, an Intercalating Carrier for the onium ion spacing agent, and a layered Phyllosilicate.

“Exfoliate” or “Exfoliated” shall mean individual platelets of an Intercalated layered Phyllosilicate so that adjacent platelets of the Intercalated layered Phyllosilicate can be dispersed individually throughout a carrier material, such as water, a polymer, an alcohol or glycol, or any other organic liquid, together with tactoids of 2-20 layers of non-exfoliated platelets.

“Exfoliation” shall mean a process for forming an Exfoliate from an Intercalate.

### Clay Purification and Ion-Exchange

A preferred layered phyllosilicate useful for interaction with an inactivation of viruses is a smectite clay that has been purified and ion-exchanged in accordance with this assignee's U.S. Pat. No. 6,050,509, hereby incorporated by reference. The ion-exchange process can be used to provide a homoionic layered phyllosilicate or can be used to provide the phyllosilicate with mixed cations from the periodic table groups 1a, 1b, 2a, 2b, 3a, 3b, 4b, 5b, 6b, 7b, 8, tin, hydrogen, lead, and/or protonated onium ions, within any percentage of the phyllosilicate exchangeable cations (1-99% of the exchangeable cations). According to U.S. Pat. No. 6,050,509 the smectite clay slurry is pumped to a series of ion exchange columns where any undesirable cation is exchanged with a desirable cation. In this manner, the crude montmorillonite clay can be exchanged to produce a purified montmorillonite with a single (homoionic) desirable cation or with a mixture of cations. In this manner, by using the appropriate ion exchange column, any element can be exchanged for the interlayer cations of a phyllosilicate for virus inactivation, including hydrogen and/or one or more elements from the following groups of the periodic table: group 1a (e.g., lithium, sodium, potassium) group 2a (e.g., magnesium, calcium, barium) group 3b (e.g., lanthanium), group 4b (e.g., titanium) group Sb (e.g., vanadium), group 6b (e.g., chromium), group 7b (e.g., manganese) group 8 (e.g., iron, cobalt, nickel, platinum), group 1b (e.g., copper, gold, silver), group 2b (e.g., zinc, cadmium) group 3a (e.g., boron, aluminum) and selected members of group 4a (e.g., tin and lead). In this manner, one could exchange a metal or metal cation with known, good antimicrobial or antiviral properties on the surface of the montmorillonite clay, or any layered phyllosilicate material, to produce a material with superior antimicrobial and antiviral properties. Homoionic hydrogen ion-exchanged layered phyllosilicates are formed as follows: (1) A slurry of 1% by weight of sodium montmorillonite clay in de-ionized water was prepared; (2) The 1% by weight sodium montmorillonite slurry was pumped through an ion-exchange column filled with hydrogen ion-exchange beads. The hydrogen ion-exchange beads were formed by contacting ion-exchange beads with an excess of 2N HCI; and (3) The hydrogen ion-exchanged slurry was diluted to 0.1% by weight for testing.

In accordance with this embodiment of the virucidal layered phyllosilicate, the crude layered phyllosilicate deposits initially include one or more of the following non-smectite impurities: (SiO₂), feldspar (KAlSi₃ O₈), opal-CT (SiO₂); gypsum (CaSO₄.2H₂O); albite (NaAlSi₃O₈); anorthite (CaAl₁₂Si₂O₈); orthoclase (KAlSi₃O₈); apatite (Ca₅ (PO₄)₃(F,Cl,OH)); halite (NaCl); calcite (CaCO₃); dolomite (CaMg(CO₃)₂; sodium carbonate (Na₂CO₃); siderite (FeCO₃) biotite (K(Mg,Fe)₃(AlSi₃O₁₀) (OH)₂) muscovite (KAl₂(AlSi₃O₁₀) (OH)₂); chlorite ((Mg,Fe)₆(Si,Al)₄O₁₀ (OH)₈); stilbite (NaCa₂Al₅Si₁₃O₃₆.14H₂O); pyrite (FeS₂); kaolinite (Al₂Si₂O₅.(OH)₄); and hematite (Fe₂O₃)

In order to remove at least 90% by weight of the above impurities, preferably at least 99% of the impurities, preferably, the layered phyllosilicate is dispersed in water, preferably at a concentration of about 10% to about 15% by weight, based on the total weight of phyllosilicate and water. The preferred layered phyllosilicate is a smectite clay, such as a montmorillonite clay, that is predominantly (greater than about 50% by weight) sodium or calcium montmorillonite clay so that the concentration of clay dispersed in water can be as high as about 15% by weight. If, for example, a sodium montmorillonite clay is dispersed in water, the higher swelling capacity of sodium montmorillonite in water will result in a viscosity that is too high for handling at a concentration of about 6-10% by weight. Accordingly, in order to achieve the most efficient purification of the smectite clay, it is preferred that the clay dispersed in water is a montmorillonite clay having predominantly (at least 50% by number) multivalent cations, i.e., Ca⁺² in the interlayer space, such as calcium montmorillonite clay. If the clay is not predominantly a multivalent clay, such as calcium montmorillonite, it can be ion-exchanged sufficiently to provide predominantly multivalent ions in the interlayer spaces between montmorillonite clay platelets.

The clay slurry is then directed into a series of cascaded hydrocyclones of decreasing size, each hydrocyclone capable of removing impurities of at least a particular size, particularly the impurities having a size greater than about 74 microns. The resulting clay, separated from the impurities, has a particle size such that at least about 90% by volume of the clay particles have a size below about 74 microns, preferably below about 50 microns, more preferably below about 20 microns. The clay slurry is then directed upwardly through a cation exchange column that removes multivalent interlayer cations from the montmorillonite clay (e.g., divalent and/or trivalent cations) and substitutes monovalent cations such as sodium, lithium and/or hydrogen for the multivalent cations within the interlayer spaces between platelets of the montmorillonite clay.

After essentially complete ion exchange, such that the clay has at least 90%, preferably at least 95%, more preferably at least 99%, by number, monovalent cations in the interlayer spaces, the clay preferably is then directed into a high speed centrifuge where the clay is subjected to centrifugal force equal to, for example, at least about 2,000 G (forces of gravity) Lip to about 4,000 G, preferably about 2,500 G to about 3,500 G, capable of removing clay particle sizes between about 5 microns and about 74 microns, such that the remaining montmorillonite clay particles, having less than about 50 by weight crystalline and amorphous non-smectite clay impurities, preferably less than about 5% by weight impurities therein, have a particle size of about 10 microns or less, preferably about 8 microns or less, and have an average particle size less than about 3 microns, preferably less than about 2 microns.

In accordance with an important feature of this embodiment, for effective removal of the impurities that have a size less than about 10 microns in diameter, the clay should first be conditioned or treated for removal of all multivalent, e.g., divalent and trivalent, interlayer cations by substitution of the multivalent cations with one or more monovalent cations, such as sodium ions, or protonated onium ions, in order to provide effective removal of the smallest impurities, for example, in a high speed (2,000-4,000 G) centrifuge. In accordance with another important feature of this embodiment, it has been found that conveying the clay slurry through the hydrocyclones prior to monovalent, e.g., sodium ion-exchange provides for a much more efficient process since the material fed to the hydrocyclones can be fed at a higher solids content without an undue increase in the viscosity of the material fed to the hydrocyclones. Accordingly, ion-exchange is accomplished after the clay slurry is passed through the hydrocyclones and before sending the partially purified clay slurry to a centrifuge for removal of the smallest impurities removed from the product.

The product from primary and secondary one inch hydrocyclones are fed by gravity to an ion-exchange feed tank where the clay/water slurry, including impurities, are maintained at a clay concentration of about 1-7% by weight, preferably about 3-7% by weight, based on the total weight of material in the ion-exchange feed tank. The clay slurry from the ion-exchange feed tank is pumped to a series of ion-exchange columns where the interlayer clay cations are exchanged with cations from periodic table groups 1a, 1b, 2a, 2b, 3a, 3b, 4b, 5b, 6b, 7b, 8, tin or lead, preferably sodium. Ion-exchange is achieved, for example, by contact with an ion-exchange resin, preferably PUROLITE C-100, obtained from The PUROLITE Company, a polystyrene cross linked with divinyl benzene, in spherical bead form, in the sodium ionic form, having an 8% by weight divinyl benzene content.

The product from a secondary one inch hydrocyclone includes at least about 90% by number particles having a size less than about 50 microns, preferably less than about 20 microns, more preferably less than about 10 microns, a mean particle size less than about 10 microns, and a median particle size less than about 5 microns.

### Exfoliated Clay to Form Clay Platelets and/or Tactoids

To form the intercalated and exfoliated layered phyllosilicates described herein, the phyllosilicate material, e.g., bentonite, should be swelled or intercalated, in the preferred embodiment, by sorption of an onium ion spacing agent.

While the compositions and methods described herein are described by way of the preferred embodiment via expanding the interlaminar spacing between adjacent platelets of a layered phyllosilicate material by intercalating onium ions between the silicate platelets, the interlaminar spacing also can be achieved by intercalating a silane coupling agent, or by an acidification technique, by substitution with hydrogen (ion-exchanging the interlayer cations with hydrogen by use of an acid or ion-exchange resin) as disclosed in the Deguchi U.S. Pat. No. 5,102,948, and in the Lan, et al. U.S. Pat. No. 5,853,886, both patents hereby incorporated by reference. In this clay exfoliation embodiment, the extremely small size of the individual platelets and clay tactoids should permit interaction with and inactivation of all viruses, including neoviruses, polioviruses type 2, enteroviruses, bovine rotavirus, and bovine corona viruses.

Sorption of the onium ion spacing agent should be sufficient to achieve expansion of the interlayer spacing of adjacent platelets of the layered phyllosilicate material (when measured dry) by at least about 3 Å, preferably at least about 5 Å.

The onium ion spacing agent is introduced into the layered phyllosilicate galleries in the form of a solid or liquid composition (neat or aqueous, with or without an organic solvent, e.g., an aliphatic hydrocarbon, such as heptane to, if necessary, aid to dissolve the onium ion compound) having an onium ion spacing agent concentration sufficient to provide a concentration of about 5% to about 10% by weight phyllosilicate (90-95% water) and the onium ion compound is dissolved in the phyllosilicate slurry water, preferably at a molar ratio of onium ions to exchangeable interlayer cations of at least about 0.25:1, more preferably at least about 0.5:1, most preferably at least about 1:1. The onium ion-intercalated layered phyllosilicate then is separated from the water easily, since the phyllosilicate is now hydrophobic, and dried in an oven to less than about 15% water, preferably bone dry, before interaction with the virus. The onium ion spacing agent compound can be added as a solid with the addition to the layered phyllosilicate material/onium ion compound blend of preferably at least about 20% water, more preferably at least about 30% water or more, based on the dry weight of layered material. Preferably about 30% to about 50% water, more preferably about 30% to about 40% water, based on the dry weight of the layered material, is included in the onium ion intercalating composition, so that less water is sorbed by the intercalate, thereby necessitating less drying energy after onium ion intercalation.

The onium ion spacing agent cations intercalated via ion-exchange into the interlayer spaces between adjacent layered material platelets are primary, secondary, tertiary or quaternary onium ions having the following preferred structure: 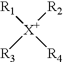
wherein X=N, P, S, or O; and
wherein R₁, R₂, R₃ and R₄ are H or organic moieties, such as linear or branched alkyl, aryl or aralkyl moieties having 1 to about 24 carbon atoms.

The more preferred protonated C₆+ onium ions are preferably quaternary ammonium ions having Formula 1, as follows:

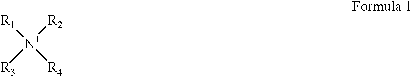

wherein R₁ is a long chain alkyl moiety ranging from C₆ to C₂₄, straight or branched chain, including mixtures of long chain moieties, i.e., C₆, C₈, C₁₀, C₁₂, C₁₄, C₁₆, C₁₈, C₂₀, C₂₂ and C₂₄, alone or in any combination; and R₂, R₃ and R₄ are moieties, same or different, selected from the group consisting of H, alkyl, benzyl, substituted benzyl, e.g., straight or branched chain alkyl-substituted and halogen-substituted; ethoxylated or propoxylated alkyl; ethoxylated or propoxylated benzyl, e.g., 1-10 moles of ethoxylation or 1-10 moles of propoxylation. Preferred protonated onium ions include protonated octadecylamine, protonated hexyl aminie; protonated octyl amine; protonated tallow amine; protonated tallow diamine; protonated tallow triamine; protonated tallow tetraamine; protonated hydrogenated tallow amine; protonated hydrogenated tallow diamine; protonated hydrogenated tallow triamine; protonated hydrogenated tallow tetraamine; protonated octadecyl amine; and mixtures thereof.
R¹—X⁺—R—Y⁺
where X⁺ and Y⁺, same or different, are ammonium, sulfonium, phosphonium, or oxonium radicals such as ⁺NH₃, ⁺NH₂—, ⁺N(CH₃)₃, ⁺N(CH₃)₂—, ⁺N(CH₃)₂(CH₂CH₃), ⁺N(CH₃)(CH₂CH₃)—, ⁺S(CH₃)₃, ⁺S(CH₃)₂—, ⁺P(CH₃)₃, ⁺P(CH₃)₂—, ⁺NH₄, ⁺NH₃—, and the like; R is an organic spacing, backbone radical, straight or branched, preferably having from 2 to 24, more preferably 3 to 10 carbon atoms, in a backbone organic spacing molecule covalently bonded at its ends to charged N⁺, P⁺, S⁺ and/or O⁺ cations and R¹ can be hydrogen, or an alkyl radical of 1 to 22 carbon atoms, linear or branched, preferably having at least 6 carbon atoms. Examples of R include substituted or unsubstituted alkylene, cycloalkenylene, cycloalkylene, arylene, alkylarylene, either unsubstituted or substituted with amino, alkylamino, dialkylamino, nitro, azido, alkenyl, alkoxy, cycloalkyl, cycloalkenyl, alkanoyl, alkylthio, alkyl, aryloxy, arylalkylamino, alkylamino, arylamino, dialkylamino, diarylamino, aryl, alkylsufinyl, aryloxy, alkylsulfinyl, alkylsulfonyl, arylthio, arylsulfinyl, alkoxycarbonyl, arylsulfonyl, or alkylsilane. Examples of R1 include non-existent; H; alkyl having 1 to 22 carbon atoms, straight chain or branched; cycloalkenyl; cycloalkyl; aryl; alkylaryl, either unsubstituted or substituted or substituted with amino, alkylamino, dialkylamino, nitro, azido, alkenyl, alkoxy, cycloalkyl, cycloalkenyl, alkanoyl, alkylthio, alkyl, aryloxy, arylalkylamino, alkylamino, arylamino, dialkylamino, diarylamino, aryl, alkylsufinyl, aryloxy, alkylsulfinyl, alkylsulfonyl, arylthio, arylsulfinyl, alkoxycarbonyl, arylsulfonyl, or alkylsilane. Illustrative of useful R groups are alkylenes, such as methylene, ethylene, octylene, nonylene, tert-butylene, neopentylene, isopropylene, sec-butylene, dodecylene and the like; alkenylenes such as 1-propenylene, 1-butenylene, 1-pentenylene, 1-hexenylene, 1-heptenylene, 1-octenylene and the like; cycloalkenylenes such as cyclohexenylene, cyclopentenylene and the like; alkanoylalkylenes such as butanoyl octadecylene, pentanoyl nonadecylene, octanoyl pentadecylene, ethanoyl undecylene, propanoyl hexadecylene and the like; alkylaminoalkylenes, such as methylamino octadecylene, ethylamino pentadecylene, butylamino nonadecylene and the like; dialkylaminoalkylene, such as dimethylamino octadecylene, methylethylamino nonadecylene and thelike; arylaminoalkylenes such as phenylamnino octadecylene, p-methylphenylamino nonadecylenie and the like; diarylaniinoalkylenes, such as diphenylamnino pentadecylene, p-nitrophelnyl-p-α-methylphenylamino octadecylene and the like; alkylarylamrinoalkylenes, such as 2-phenyl-4-methylamino pentadecylene and the like; alkylsulfinylenes, alkylsul fonylenes, alkylthio, arylthio, arylsulfinylenes, and arylsulfonylenes such as butylthio octadecylene, neopentylthio pentadecylene, methylsulfinyl nonadecylene, benzylsulfinyl pentadecylene, phenylsulfinyl octadecylene, propylthiooctadecylene, octylthio pentadecylene, rionylsulfonyl nonadecylene, octylsulfonyl hexadecylene, methylthio nonadecylene, isopropylthio octadecylene, phenylsulfonyl pentadecylene, methylsulfonyl nonadecylene, nonylthio pentadecylene, phenylthio octadecylene, ethyltio nonadecylene, benzylthio undecylene, phenethylthio pentadecylene, sec-butylthio octadecylene, naphthylthio undecylene and the like; alkoxycarbonylalkylenes such as methoxycarbonylene, ethoxycarbonylene, butoxycarbonylene and the like; cycloalkylenes such as cyclohexylene, cyclopentylene, cyclo-octylene, cycloheptylene and the like; alkoxyalkylenes such as methoxy-methylene, ethoxymethylene, butoxymethylene, propoxyethylene, pentoxybutylene and the like; aryloxyalkylenes and aryloxyarylenes such as phenoxyphenylene, phenoxymethylene and the like; aryloryalkylenes such as phenoxydecylene, phenoxyoctylene and the like; arylalkylenes such as benzylene, phenthylene, 8-phenyloctylene, 10-phenyldecylene and the like; alkylarylenes such as 3-decylphenylene, 4-octylphenylene, 4-nonylphenylene and the like; and polypropylene glycol and polyethylene glycol substituents such as ethylene, propylene, butylene, phenylene, benzylene, tolylene, p-styrylene, p-phenylmethylene, octylene, dodecylene, octadecylene, methoxy-ethylene, moieties of the formula —C₃H₆COO—, —C₅H₁₀COO—, —C₇H₁₀COO—, —C₇H₁₄COO—, —C₉H₁₈COO—, —C₁₁H₂₂COO—, —C₁₃H₂₆COO—, —C₁₅H₃₀COO—, and —C₁₇H₃₄COO— and —C═C(CH₃)COOCH₂CH₂—, and the like. Such tetra-, tri-, and di-ammonium, -sulfonium, -phosphonium, -oxonium; ammonium/sulfonium; ammonium/phosphonium; ammonium/oxonium; phosphonium/oxonium; sulfonium/oxonium; and sulfonium/phosphonium radicals are well known in the art and can be derived from the corresponding amines, phosphines, alcohols or ethers, and sulfides.

Other useful spacing agent compounds are multi-onium ion compounds that include at least two primary, secondary, tertiary or quaternary ammonium, phosphonium, sulfonium, and/or oxonium ions having Formula 2, as follows: 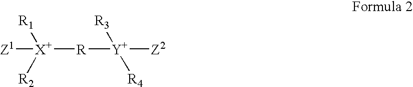
wherein R is an alkylene, aralkylene or substituted alkylene charged atom spacing moiety, preferably ranging from C₃ to C₂₄, more preferably about C₃ to C₆ for relatively high charge density (150 milliequivalents/100 grams C.E.C. to 70 milliequivalents/100 grams C.E.C.) layered materials; and preferably from C₆ to C₁₂ for medium to low charge density (70 milliequivalents/100 grams C.E.C. to 30 milliequivalents/100 grams C.E.C.) layered materials. R can be straight or branched chain, including mixtures of such moieties, i.e., C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃ and C₂₄, alone or in any combination; and R₁, R₂, R₃ and R₄ are moieties, same or different, selected from the group consisting of hydrogen, alkyl, aralkyl, benzyl, substituted benzyl, e.g., straight or branched chain alkyl-substituted and halogen-substituted; ethoxylated or propoxylated alkyl; ethoxylated or propoxylated benzyl, e.g., 1-10 moles of ethoxylation or 1-10 moles of propoxylation. Z¹ and Z², same or different, may be non-existent, or may be any of the moieties described for R₁, R₂, R₃ or R₄. Also, one or both of Z¹ and Z² may include one or more positively charged atoms or onium ion molecules.

Any swellable layered phyllosilicate material that sufficiently sorbs the onium ion spacing agent to increase the interlayer spacing between adjacent phyllosilicate platelets by at least about 3 Å, preferably at least about 5 Å, can be used in the practice of this invention. Useful swellable layered materials include phyllosilicates, such as smectite clay minerals, e.g., montmorillonite, particularly sodium montmorillonite, magnesium montmorillonite and/or calcium montmorillonite; nontronite; beidellite; laponite; yakhontovite; zincsilite; volkonskoite; hectorite; saponite; ferrosaponite; sauconite; swinefordite; pimelite; sobockite; stevensite; svinfordite; vermiculite; synthetic clays; mixed layered illite/smectite minerals, such as rectorite, tarosovite, and ledikite; admixtures of illites with the clay minerals named above, magnesium aluminum silicates; ion-exchanged phyllosilicates, including homoionic and/or protonated phyllosilicates; and mixtures of any two or more of the above-listed phyllosilicates. Exemplary mixtures include any of the above-listed phyllosilicates, wherein one of the above-listed phyllosilicates is present in amount ranging from about 1%-99% wt. and another phyllosilicate is present in an amount ranging from 99%-1% wt.; or wherein one of the above-listed phyllosilicates is present in amount greater than 50% wt and another phyllosilicate is present in an amount less than 50% wt; or wherein one of the above-listed phyllosilicates is present in amount of 50% wt and a second phyllosilicate is present in an amount of 50%; or wherein one of the above-listed phyllosilicates is present in amount of about 10% wt and another phyllosilicate is present in an amount of about 90%; or wherein one of the above-listed phyllosilicates is present in amount of about 20% wt and another phyllosilicate is present in an amount of about 80%; or wherein one of the above-listed phyllosilicates is present in amount of about 30% wt and another phyllosilicate is present in an amount of about 70% wt; or wherein one of the above-listed phyllosilicates is present in amount of about 40% wt and another phyllosilicate is present in an amount of about 60% wt. The weight percent indicated above is based on the weight of the clay mixture.

Preferred swellable layered materials are phyllosilicates of the 2:1 type having a negative charge on the layers ranging from about 0.15 to about 0.9 charges per formula unit and a commensurate number of exchangeable metal cations in the interlayer spaces. Most preferred layered materials are smectite clay minerals such as montmorillonite, nontronite, beidellite, volkonskoite, hectorite, saponite, sauconite, sobockite, stevensite, and svinfordite.

As used herein the “interlayer spacing” refers to the distance between the internal faces of the adjacent phyllosilicate layers as they are assembled in the layered material before any delamination (exfoliation) takes place. The preferred clay materials generally include interlayer cations such as Na⁺, Ca⁺², K⁺, Mg⁺², Al⁺³⁺, NH₄ and the like, including mixtures thereof, and can be ion-exchanged to include other cations such as the elements from period table group 1a, 1b, 2a, 2b, 3a, 3b, 4b, 5b, 6b, 7b, 8, tin and lead.

The onium ions, may be introduced into (sorbed within) the interlayer spaces of the layered phyllosilicate in a number of ways. In a preferred method of intercalating the onium ions between adjacent platelets of the layered material, the phyllosilicate material is slurried in water, e.g., at 5-20% by weight layered phyllosilicate material and 80-95% by weight water, and the onium ion compound is dissolved in the water in which the phyllosilicate material is slurried. If necessary, the onium ion compound can be dissolved first in an organic solvent, e.g., propanol. The phyllosilicate material then is separated from the slurry water and dried suspending the individual silicate platelets and tactoids in a liquid carrier.

To achieve sufficient intercalation of the onium ions between adjacent platelets of the layered phyllosilicate, the phyllosilicate/onium ion intercalating composition preferably contains a molar ratio of onium ions to layered phyllosilicate of at least 0.25: 1, more preferably at least 0.5:1 for the onium ions to exchange interlayer cations with the smectite clay, most preferably 1:1, based on the dry weight of the phyllosilicate, so that the resulting onium ion-intercalated phyllosilicate has interior platelet surfaces that are sufficiently hydrophobic and sufficiently spaced for exfoliation and suspension of the individual platelets and tactoids in a liquid carrier. The onium ion carrier (preferably water, with or without an organic solvent) can be added by first solubilizing or dispersing the onium ion compound in the carrier; or a dry onium ion compound and relatively dry layered phyllosilicate (preferably containing at least about 4% by weight water) can be blended and the intercalating carrier added to the blend, or to the phyllosilicate prior to adding the dry onium ion. When intercalating the phyllosilicate with onium ions in slurry form, the amount of water can vary substantially, e.g., from about 4% by weight, preferably from a minimum of at least about 30% by weight water, with no upper limit to the amount of water in the intercalating composition (the phyllosilicate intercalate is easily separated from the intercalating composition due to its hydrophobicity after onium ion treatment).

Alternatively, the onium ion intercalating carrier, e.g., water, with or without an organic solvent, can be added directly to the phyllosilicate prior to adding the onium ion compound, either dry or in solution. Sorption of the onium ion compound molecules may be performed by exposing the phyllosilicate to a dry or liquid onium ion compound in the onium ion intercalating composition containing at least about 2% by weight, preferably at least about 5% by weight onium ion compound, more preferably at least about 10% onium ion compound, based on the dry weight of the layered phyllosilicate material.

In accordance with an emulsion method of intercalating the onium ions between the platelets of the layered phyllosilicate material, the phyllosilicate, preferably containing at least about 4% by weight water, more preferably about 10% to about 15% by weight water, is blended with water and/or organic solvent solution of an onium ion spacing agent compound in a ratio sufficient to provide at least about 5% by weight, preferably at least about 10% by weight onium ion compound, based on the dry weight of the layered phyllosilicate material.

The onium ion spacing agents have an affinity for the phyllosilicate so that they are sorbed between, and are ion-exchanged with the cations on the inner surfaces of the silicate platelets, in the interlayer spaces.

### PROTONATED ONIUM ION INTERCALATION EXAMPLES:

### Example 1

Example 1 demonstrates the ion exchange process of smectite clay from a Ca form or Na/Ca mixed forms to Na-rich smectite clay.

Raw smectite clay was dispersed into water to make a 3 wt % clay slurry. This clay has a Na content of 0.20 wt % and Ca content of2.10 wt %. The elemental analysis was measured by an X-ray fluorescence method. The mixture was mixed thoroughly with a mechanical mixer. The pH value of the starting clay slurry is 7-8. An ion exchange resin, such as Amberlite 200C Na, is available from Rohm & Hass packed in a glass column with a 2-in diameter and a 20-in length. A liquid pump was used to pump the clay slurry through the column at 20 ml/min. Elemental analysis of the finished clay, dried from the slurry, indicated that the Na content is 3.45 wt % and Ca content is 0.17 wt %. The ion exchanged clay is called El-Na-Clay. This clay had a basal spacing of 13 Å.

### Example 2

Example 2 demonstrates the formation of protonated Octadecyl ammonium-treated smectite clay with Octadecyl ammonium acetate from the ion exchanged Na-smectite clay (El-Na-clay) of Example 1.

100-g of sodium smectite clay El-Na-clay was dispersed into 3000 ml water through a mechanical mixer. This clay slurry was heated to 80° C. 41.5 g of Octadecyl ammonium acetate from KAO Chemicals was added into the clay slurry. The clay showed excellent flocculation after the addition of the Octadecyl ammonium acetate. The pH of the clay reaction slurry was about 4. The clay was filtered with regular quantitative filter paper with the assistance of a mechanical vacuum pump. Then, the clay was dried in an oven over night at 80° C. and ground to pass through a 300-mesh screen as a fine powder. This modified clay was called E2-ODA-Clay.

### Example 3

Example 3 demonstrates the formation of protonated Octadecyl ammonium-treated smectite clay with a solution of Octadecyl ammonium ions in dilute HCI. (E3-ODA-Clay). This sample was measured by powder X-ray diffraction to determine the clay basal spacing after ion exchange. The result is listed in Table-1.

100-g of sodium smectite E1-Na-clay was dispersed into 3000 ml water through a mechanical mixer. This clay slurry was heated to 80° C. 33.8 g of Octadecyl amine was added into 1000 ml of 70° C. water and then mixed with 17.1 g of 10.5 N HCI. The Octadecyl amine-HCI solution was added into the clay slurry followed by mixing. The reaction slurry had a pH of 4. The clay showed excellent flocculation after the addition of the Octadecyl amine-HCI solution. The clay was filtered with regular quantitative filter paper with the assistance of a mechanical vacuum pump. Then, the clay was dried in an oven over night at 80° C. and ground to pass through a 300-mesh screen as a fine powder. This modified clay was called E3-ODA-Clay. This sample was measured by powder X-ray diffraction to determine the clay basal spacing after ion exchange. The result is listed in Table-1.

### Viruses and Viral Taxonomy

Viruses constitute a large and heterogeneous group, and they are classified in hierarchical taxonomic categories based on many different characteristics, e.g., morphology, antigenic properties, physiochemical and physical properties, proteins, lipids, carbohydrates, molecular properties, organization and replication, and biological properties. Whether the RNA or DNA is single or double stranded, the organization of the genome and the presence of particular genes comprise important aspects of the current taxonomy of viruses. All of the former are used to place a virus into a particular order or family. The classification is based upon macromolecules produced (structural proteins and enzymes), antigenic properties and biological properties (e.g., accumulation of virions in cells, infectivity, hemagglutination).

Viral classification is dynamic in that new viruses are continuously being discovered and more information is accumulating about viruses already known. The classification and nomenclature of the latest known viruses appear in reports of the International Committee on the Taxonomy of Viruses (ICTV), 7th edition (van Regenmortel et al., editors. Seventh ICTV report. San Diego: Academic Press; 2000.) The basic viral hierarchical classification scheme is: Order, Family, Subfamily, Genus, Species, Strain, and Type as set out below.

Virus orders represent groupings of families of viruses that share common characteristics and are distinct from other orders and families. Virus orders are designated by names with the suffix-virales. Virus families are designated by names with the suffix-viridae. Virus families represent groupings of genera of viruses that share common characteristics and are distinct from the member viruses of other families. Viruses are placed in families on the basis of many features. A basic characteristic is nucleic acid type (DNA or RNA) and morphology, that is, the virion size, shape, and the presence or absence of an envelope. The host range and immunological properties (serotypes) of the virus are also used. Physical and physicochemical properties such as molecular mass, buoyant density, thermal inactivation, pH stability, and sensitivity to various solvents are used in classification. Virus genera represent groupings of species of viruses that share common characteristics and are distinct from the member viruses of other genera. Virus genera are designated by terms with the suffix-virus. A virus species is defined as a polythetic class of viruses that constitutes a replicating lineage and occupies a particular ecological niche.

Some viral families and their respective, sub-families, genera, and species contemplated for inactivation by contact and adsorption by the clays described herein include, but are not limited to, the following viruses set out in Tables 1-3 below. The preferred viruses inactivated by contact and adsorption by the clays described herein are any or all of the viruses set out in Tables 1-3 except for Reoviridae and its genera rotavirus; poliovirus type 2; enteroviruses; bovine rotavirus; and bovine coronaviruses.

TABLE 1
DNA VIRUSES

| Family | Sub-Family | Genus | Virus |
|---|---|---|---|
| Herpesviridae | Alphaherpes- | Simplexvirus | Herpes simplex |
| | virinae | | type 1 (HHV-1) |
| | | | Herpes simplex |
| | | | type 2 (HHV-2) |
| | | Varicellovirus | Varicella zoster |
| | | | virus (HHV-3) |
| | Betaherpes- | Cytomegalovirus | Cytomegalovirus |
| | virinae | | virus (HHV-5) |
| | | Roseolovirus | Human herpes |
| | | | virus type 6, 7 |
| | Gammaherpes- | Lymphocrypto- | Epstein Barr |
| | virinae | virus | virus (HHV-4) |
| | | Rhadinovirus | Human herpes |
| | | | virus type 8 |

| | | |
|---|---|---|
| Poxviridae | Orthopoxvirus | Variola virus |
| | Molluscipoxvirus | Molluscum |
| | | contagiousum virus |
| Adenoviridae | Mastadenovirus | Human adenovirus |
| Papovaviridae | Papillomavirus | Papillomavirus |
| | Polyomavirus | BK virus |
| | | JC virus |
| Parvoviridae | Erythrovirus | Human parvovirus |
| | | (B19) |

TABLE 2
RNA VIRUSES

| Family | Genus | Virus |
|---|---|---|
| Picornaviridae | Rhinovirus | Rhinovirus |
| | Hepatovirus | Hepatitis A virus |
| | Rubivirus | Rubella virus |
| | Alphavirus | Eastern equine encephalitis virus |
| | Rhadinovirus | Human herpes virus type 8 |
| Togaviridae | Flavivirus | Yellow fever virus |
| | | Dengue virus |
| | | West Nile virus |
| Flaviviridae | Hepacvirus | Hepatitis C virus |
| | Coronavirus | Human coronavirus |
| | Calicivirus | Norwalk virus |
| | Rubulavirus | Mumps virus |
| Coronaviridae | Morbillivirus | Measles virus |
| Caliciviridae | Pneumovirus | Respiratory syncitial virus (RSV) |
| Paramyxoviridae | Paramyxovirus | Human parainfluenza virus 1 |
| | Lyssavirus | Rabies virus |
| | Filovirus | Ebola virus |
| | Arenavirus | Lassa fever virus |
| Rhabdoviridae | Influenzavirus A | Influenza A |
| Filoviridae | Influenzavirus B | Influenza B |
| Arenaviridae | Influenzavirus C | Influenza C |
| Orthomyxoviridae | Hantavirus | Sin Nombre virus |
| Bunyaviridae | | |

TABLE 3
DNA-RNA REVERSE TRANSCRIBING VIRUSES

| Family | Genus | Virus |
|---|---|---|
| Retroviridae | Lentivirus | Human immunodeficiency viruses |
| | BLV-HTLV | Human T-cell leukemia viruses |
| | retroviruses | |
| Hepadnaviridae | Orthohepadnavirus | Hepatitis B virus |

Uses for the Layered Phyllosilicate Material

In yet another embodiment, the invention provides various methods of using the layered phyllosilicates of the invention. In one aspect, methods of inhibiting transfer of a virus are provided. For example, provided herein is a method for inhibiting the transfer of a virus to a surface comprising contacting the surface with a composition comprising a layered phyllosilicate material in an amount sufficient for inhibiting the transfer of the virus thereto. In some aspects, the surface is an inanimate surface. Exemplary inanimate surfaces include metal surfaces (including stainless steel), glass surfaces (including pyrex), plastic surfaces (including polystyrene) and stone surfaces. In other aspects, the surface is an animate surface. Exemplary animate surfaces include bone, skin and mucous membranes.

The animate surface can be from a mammalian subject. In one aspect, the mammalian subject is an animal. Exemplary animals include, but are not limited to, farm animals such as horses, cows, sheep, pigs, llamas, alpacas and goats; companion animals such as dogs and cats; exotic and/or zoo animals; laboratory animals including mice, rats, rabbits, guinea pigs and hamsters. In other aspects, the mammalian subject is a human.

In one aspect, a layered phyllosilicate material is useful for inactivating viruses found on both animate and inanimate surfaces. Thus, in one embodiment, the invention includes methods of inactivating viruses on animate surfaces by contacting the virus with a layered phyllosilicate material. Some of the benefits of the layered phyllosilicate material include it being a natural product and it will leave no harmful residues on biological surfaces and/or will not exhibit any unwanted side effects.

In one aspect, inactivation of a virus using the layered phyllosilicate material described herein is by one or more mechanisms selected from the group consisting of adsorption, ionic complexing, electrostatic complexing, chelation, hydrogen bonding, ion-dipole, dipole/dipole, Van Der Waals forces, and any combination thereof. Such ionic bonding provides inactivation of a virus molecule by a phyllosilicate material. Viral inactivation prevents a virus from migrating to and penetrating cell membranes, thereby preventing the virus from reproducing and rupturing the cells and releasing more of the virus to attach to and infect host cells. Accordingly, the layered phyllosilicate material inhibits virus entry and fusion to host cells and provides a physical barrier between a virus and a host cell.

The use of the layered phyllosilicate material described herein for the inactivation of both enveloped and non-enveloped viruses is contemplated. An enveloped virus comprises a capsid surrounded by a lipid bilayer derived from a membrane of the host cell and membrane proteins involved in adsorption found in the envelope. Non-enveloped viruses lack this lipid bilayer surrounding the capsid and have the proteins associated with adsorption found directly on (or part of) the capsid. Because the layered phyllosilicate material interacts directly with the oppositely charged surface of a virus, the presence of the lipid envelope on an enveloped virus is not expected to affect this interaction. The oppositely charged molecules on the surface of a virus include proteins, glycoproteins, lipids and combinations thereof. Further, because the layered phyllosilicate material interacts with the oppositely charged molecules on the surface of a virus, and not the genetic material in the nucleus of the virus, the inactivation of a virus by the layered phyllosilicate material is not affected by mutation, antigenic drift, or genetic recombination of the virus. Accordingly, a method of preventing a virus from becoming resistant to a particular material, comprising contacting a virus with a material that interacts with the oppositely charged molecules of the virus is specifically contemplated. In one aspect, the interaction between the layered phyllosilicate material and the oppositely charged molecules of the virus is a mechanism selected from the group consisting of adsorption, ionic complexing, electrostatic complexing, chelation, hydrogen bonding, ion-dipole, dipole/dipole, Van Der Waals forces and combinations thereof.

One mode of application of the virucidal compositions of the invention is as a topical agent. Preferably, the topical agent is a solution, that is, a liquid formulation comprising the layered phyllosilicate material and a carrier. Other suitable forms include semi-solid or solid forms comprising a carrier for topical application and having a dynamic viscosity preferably greater than that of water, provided that the carrier does not deleteriously react with the layered phyllosilicate material in the composition. Suitable formulations include, but are not limited to, lip balms, suspensions, emulsions, creams, ointments, gels, powders, liniments, salves and the like. If desired, these formulations may be sterilized or mixed with auxiliary agents, including but not limited to, preservatives, stabilizers, wetting agents, buffers or salts for influencing osmotic pressure and the like well known in the art. Preferred vehicles for semi-solid or solid form topical preparations include ointment bases, including polyethylene glycol-1000 (PEG-1000); conventional ophthalmic vehicles; creams, (including HEB cream); and gels, (including K-Y gel, Miglyole® Gel B, Miglyol® Gel T, and Miglyol® 840 Gel B); as well as petroleum jelly and the like. These topical preparations may also contain emollients, perfumes, and/or pigments to enhance their acceptability for various usages, provided that the additives do not deleteriously react with the layered phyllosilicate material in the composition.

Also suitable for topical application are sprayable aerosol preparations wherein the layered phyllosilicate material, preferably in combination with a solid or liquid inert carrier material, is packaged in a squeeze bottle or in admixture with a pressurized volatile, normally gaseous propellant, including a Freon (chlorofluorocarbon) or environmentally acceptable volatile propellant. Such compositions can be used for application to inanimate environmental surfaces, including examining tables, toilet seats and the like, and/or for application to animate surfaces such as the skin or to mucous membranes. The aerosol or spray preparations can contain solvents, buffers, surfactants, perfumes, and/or antioxidants in addition to the layered phyllosilicate material of the invention.

In one aspect, the compositions are employed in mixture with conventional excipients, i.e., pharmaceutically acceptable organic or inorganic carrier substances suitable for topical application which do not deleteriously react with the acid or the alcohol in the composition. Suitable pharmaceutically acceptable carriers include but are not limited to water, salt solutions, alcohols, vegetable oils, polyethylene glycols, gelatin, lactose, amylose, magnesium stearate, talc, silicic acid, viscous paraffin, perfume oil, fatty acid monoglycerides and diglycerides, pentaerythritol fatty acid esters, hydroxymethylcellulose, polyvinyl pyrrolidone, lubricants, preservatives, stabilizers, wetting agents, emulsifiers, salts for influencing osmotic pressure, buffers, coloring, flavoring and/or aromatic substances and the like which do not deleteriously react with the layered phyllosilicate material in the composition.

The virucidal compositions of this invention may also be used to prevent the spread of infection by viruses that reside in, are transmitted by and/or infect the cells of the dermis or epidermis. That is, in another embodiment, the layered phyllosilicate material may be incorporated into a hand cream, gel or lotion for use by people exposed to viruses. For example, medical personnel could apply the hand cream, gel or lotion (incorporating the layered phyllosilicate material) both before and after the examination of patients with suspected virus infections. In one aspect, the supplemented hand cream, gel or lotion is for human use. In other aspects, it can be applied to animals at risk for contracting a viral infection.

In another aspect, the layered phyllosilicate material is in fluids used to kill viruses on examining tables, instruments, gloves, towels and any other inanimate surfaces which may come in contact with virus particles.

In another aspect, the composition also is formulated as a dispersable powder for dusting the skin, hair, fur, or feathers of humans or animals. The compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents and scent enhancers.

In another aspect, the layered phyllosilicate material is used in wound healing aspects to treat humans or socially or economically important animal species such as dogs, cats, horses, sheep, cows, goats, or pigs. Methods according to the present invention are not limited to use in humans.

A composition comprising the layered phyllosilicate material is suitable for use in situations in which wound healing is required. Exemplary situations include, but are not limited to: (1) diabetic foot and leg ulcerations, including neuropathic ulcerations, decubitus lesions, and necrobiosis lipoidica diabeticorum; (2) vascular ulcerations, including venous stasis ulceration, arterial ulcerations, varicose vein ulcerations, post-thrombotic ulcerations, atrophie blanche ulcerations, congenital absence of veins/ulcerations, congenital or traumatic arteriovenous anastomosis, temporal arteritis, atherosclerosis, hypertension, thrombosis, embolism, platelet agglutination, ankle blow-out syndrome, or hemangiomas; (3) decubitus ulcers or pressure source; (4) traumatic ulcerations, such as those caused by external injuries, bums, scalds, chemical injuries, post-surgical injuries, self-inflicted injuries, lesions at an injection site, neonatal or perinatal trauma, or sucking blisters; (5) infestations and bites, such as those caused by spiders, scorpions, snakes, or fly larvae; (6) cold injury, such as perniosis (erythrocyanosis frigida), or cryoglobulinemic ulcerations; (7) neoplastic ulceration, such as those caused by basal cell carcinomas, squamous cell carcinomas, malignant melanomas, lymphoma, leukemia, Kaposi's sarcoma, tumor erosion, midline lethal granuloma, or Wegener's granulomatosis; (8) blood diseases with ulcerations, such as polycythemia, spherocytosis, or sickle cell anemia; (9) skin diseases with ulcerations, such as tinea, psoriasis, pemphigoid, pemphigus, neurotic excoriations, trichotillomania, erosive lichen planus, or chronic bullous dermatosis of childhood; (10) metabolic disease ulcerations, such as those associated with diabetes mellitus or gout (hyperuricemia); (11) neuropathic ulcerations, such as those associated with diabetes mellitus, tabes dorsalis, or syringomyelia; (12) ischemic ulcerations, such as those associated with scars, fibrosis, or radiation dermatitis; (13) vasculitis ulcerations, such as those associated with lupus erythematosus, rheumatoid arthritis, scleroderma, immune complex disease, pyoderma gangrenosum, or ulceration associated with lipodermatosclerosis; (14) infectious ulcerations, such as: (a) viral ulcerations, e.g. those associated with Herpes simplex or Herpes zoster in an immunocompromised or normal individual; (b) bacterial infections with ulcerations, such as those associated with tuberculosis, leprosy, swimming pool granuloma, ulceration over osteomyelitis, Buruli ulcer, gas gangrene, Meleny's ulcer, bacterial gangrene associated with other bacterial infection, scalded skin syndrome, ecthyma gangrenosum, and toxic epidermal necrolysis; (c) mycotic ulcerations, such as those associated with superficial fungal infection or deep fungal infection; (d) spirochetal ulcerations, such as those associated with syphilis or yaws: (e) leishmaniasis; (f) mydriasis; or (g) cellulitis; (15) surgical ulcerations, such as those associated with closed incisions or excisions, open incisions or excisions, stab wounds, necrotic incisions or excisions, skin grafts, or donor sites; or (16) other ulcerations, such as those associated with skin tears (traumatic ulcerations), fistula, peristomal ulcerations, ulcerations associated with aplasia cutis congenita, ulcerations associated with epidermolysis bullosa, ulcerations associated with ectodermal dysplasias, ulcerations associated with congenital protein C or S deficiency, ulcerations associated with congenital erosive and vesicular dermatosis, ulcerations associated with acrodermatitis enteropathica, and amputation stump ulcerations. The layered phyllosilicate material of the present invention can also be used to promote wound healing in other conditions.

In another aspect, a composition comprising the layered phyllosilicate material will further comprise a therapeutic agent. The therapeutic agent may be selected from the group consisting of anti-inflammatory agents, including hydrocortisone, prednisone, and the like; NSAIDS, including acetaminophen, salicylic acid, ibuprofen, and the like; selective COX-2 enzyme inhibitors, antibacterial agents, including colloidal silver, penicillill, erythromycin, polymyxin B, viomycin, chloromycetin, streptomycins, cefazolin, ampicillin, azactam, tobrarnycin, cephalosporins, bacitracin, tetracycline, doxycycline, gentamycin, quinolines, neomycin, clindamycin, kanamycin, metronidazole, and the like; antiparasitic agents including quinacrine, chloroquine, vidarabine, and the like; antifungal agents including nystatin, and the like; anti-virucides, and antiviral agents including acyclovir, docosanol, ribarivin, interferons, and the like; cellulose acetate, carbopol and carrageenan (CAS No. 9000-07-1); systemic analgesic agents including salicylic acid, acetaminophen, ibuprofen, naproxen, piroxicam, flurbiprofen, morphine, and the like; local anesthetics including cocaine, lidocaine, bupivacaine, xylocaine, benzocaine, and the like; an antisense nucleotide, a thrombin inhibitor, an antithrombogenic agent, a tissue plasminogen activator, a thrombolytic agent, a fibrinolytic agent, a vasospasm inhibitor, a calcium channel blocker, a nitrate, a nitric oxide promoter, a vasodilator, an antimicrobial agent, an antibiotic, an anti-platelet agent, an anti-mitotic, a microtubule inhibitor, an actin inhibitor, a remodeling inhibitor, an agent for molecular genetic intervention, a cell cycle inhibitor, an inhibitor of the surface glycoprotein receptor, an anti-metabolite, an anti-proliferative agent, an anti-cancer chemotherapeutic agent, an anti-inflammatory steroid, an immunosuppressive agent, an antibiotic, a radiotherapeutic agent, iodine-containing compounds, barium-containing compounds, a heavy metal functioning as a radiopaque agent, a peptide, a protein, an enzyme, an extracellular matrix component, a cellular component, a biologic agent, an angiotensin converting enzyme (ACE) inhibitor, ascorbic acid, a free radical scavenger, an iron chelator, an antioxidant, a radiolabelled form or other radiolabelled form of any of the foregoing, or a mixture of any of these. Compositions comprising a layered phyllosilicate material and an antiviral agent (including acyclovir, docosanol, ribarvirin, oseltamivir phosphate and interferons) are specifically contemplated.

In yet another aspect, the layered phyllosilicate material of the invention is present in any of the formulations described herein in a concentration (w/v) ranging from about 0.01% to about 20%, or from about 0.1% to about 10%, or in a concentration of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19% or about 20%.

Kits and Unit Doses

In related variations of the preceding embodiments, a composition comprising a layered phyllosilicate material may be so arranged, e.g., in a kit or package or unit dose, to permit co-administration with one or more other therapeutic agents, but the layered phyllosilicate material composition and the agent are not in admixture. In another aspect, the layered phyllosilicate material composition and the agent are in admixture. In some embodiments, the two components to the kit/unit dose are packaged with instructions for administering the two agents to a subject for treatment of one of the above-indicated disorders and diseases. The kit may comprise the composition of the invention in combination with a vehicle in a cream or gel base, as a pump-spray, as an aerosol, on an impregnated bandage, a medicated animal ear tag or collar, or in a dropper. In one aspect, the kit includes applicator for administering the composition. The composition of the invention may also be in any one of the above formulations in combination with a second agent, including but not limited to antibacterial agents (including colloid silver), antiviral agents, topical steroids, aloe vera and the like cosmeceuticals, Aerosil®, Cab-O-Sil® and Miglyol® Gels (including Miglyol® Gel B, Miglyol® Gel T, and Miglyol® 840 Gel B, SASOL Germany GmbH).

Other aspects and advantages of the present invention will be understood upon consideration of the following illustrative examples, which are not intended to be limiting in any way.

### Examples

### Example 4

### Antiviral Activity of Test Compounds Against HIV-1

In this study, three different compositions of bentonite clay were studied (R-0088, R-0089, and R-0090) to evaluate their adsorption and antiviral efficacy against an HIV-1 virus (Retroscreen Virology Ltd). Each bentonite clay composition was studied at three different concentrations (0.01% w/v, 0.001% w/v, and 0.0001% w/v) prepared in sterile double-distilled water) and at three different incubation times (1 minute, 5 minutes, and 10 minutes). Test compositions composed of various mineral clays and controls (as listed below) were prepared.
R-0088—purified homoionic sodium bentonite mixture, purified in accordance with U.S. Pat. No. 6,050,509.
R-0089—purified acid activated clay mixture.
R-0091—purified bentonite:dextran analog modified mixture.
C8166 growth media (negative control)
20% Ethanol/PBS (positive control)

HIV-1IIIB (AL307 with a titer of 104TCID5O/ml) was supplied from the Retroscreen Virology Ltd virus repository. Virucidal and P24 assays were carried out as set out below to evaluate antiviral activity. The p24 antigen assay measures the viral capsid (core) p24 protein in blood that is detectable earlier than HIV antibody during acute infection.

Virucidal Assay

1. 40 μl of the viral stock solution was added to each concentration of test compound (360 μl) and left to incubate at room temperature for the incubation times specified above.
2. The reaction was termninated by the addition of cell infection media (3.6 ml), which diluted the reaction 10-fold.
   P24 Assay
1. The samples were left to settle for 1.5 hours before being added to the P24 antigen coated plates.
2. 200 μl of each sample was added to the assay plate.
3. 110 μl of neat stock virus (AL307) was added to the relevant wells on the plate.
4. Empigen (final concentration of 0.8%) was added to all these wells.
5. The neat stock virus was titrated across the wells following a 10-fold dilution series in RPMI-1640 containing 1% Empigen.
6. The P24 assay was then conducted as instructed in the current Retroscreen Virology Ltd. SOP.

Only R-0088 at 0.01% w/v concentration reduced the viral titer of HIV-1_{IIIB} at the 10 minute incubation time with 99.13% efficacy exhibited. Virucidal results for R-0088 demonstrated that a time-response is exhibited by the 0.01% w/v concentration. At this concentration, the reduction in the HIV1_{IIIB} virus titer was significant at the 10 minute incubation time with a reduction of 2.29 logs. A reduction of ≧1-log₁₀ TCID₅₀/ml (Oxford et al, Anitiv. Chem. Chemother. 5:176-181, 1994) is deemed significant for the virucidal assays used in this study, and is equivalent to ≧90% reduction in viral titer. Virucidal results for R-0089 and R-0091 did not demonstrate significant reductions in HIV-1IIIB titer.

At the highest test concentration (0.01% w/v), R-0088 exhibited a significant reduction in the HIV-1_{IIIB} (AL307 with a titer of 10⁴TCID₅₀/ml). R-0089 and R-0091 did not exhibit significant reductions in the HIV1_{IIIB} virus titer for any of the variables tested.

### Example 5

### Antiviral Activity of Test Compounds Against Influenza A

This study was performed to determine whether the test compounds have virucidal efficacy against an epidemic strain of Influenza A virus and to assess the cytotoxic potential of the test compounds on Madin-Darby canine kidney cells (MDCK) cells. Three different compositions of bentonite clay (R-0088, R-0089, and R-0090) were studied to evaluate their adsorption and antiviral efficacy against an Influenza A/Panama/2007/99 (H3N2) virus.

Test compositions composed of various mineral clays and controls (as listed below) were prepared.
R-0088—purified sodium bentonite mixture, purified in accordance with U.S. Pat. No. 6,050,509.
R-0089—purified acid activated clay mixture.
R-0090—purified bentonite-sialic acid mixture.
C8166 growth media (negative control)
20% Ethanol/PBS (positive control)

Each bentonite clay mixture was studied at three different concentrations (0.01% w/v, 0.001% w/v, and 0.0001% w/v prepared in sterile double-distilled water) and at five different incubation times (30 seconds, 1 minute, 5 minutes, 10 minutes, and 30 minutes).

The cells of the toxicity controls were incubated with cell maintenance media, whereas the cells of the virucidal controls were incubated with cell infection media. The stock titer of Influenza A/Panama/2007/99 virus was 7.7 log₁₀ TCTD₅₀/ml. Before use in the virucidal assay, the stock virus was diluted 100-fold in infection media. It was then diluted a further 2-fold when it was added to the reaction mixture (section 9.3.2, step 4). The resulting test titer was therefore 5.4 log₁₀ TCID₅₀/ml. The protocols for the toxicity assay and the virucidal assay are set out below.

Toxicity assay

1. Cells (100 μl/well) at 1×10⁵ cells/ml were seeded onto 96-well plates and incubated at 37° C. for˜24 hours.
2. The cell maintenance media on the plates was removed and the cell monolayer washed twice with PBS (100 μl/well).
3. Each test compound (100 μl/well) was added, in quadruplicate, to the plate and left to incubate at room temperature for the various times specified.
4. The test compounds were removed and the cell monolayer washed twice with phosphate buffered saline (PBS) (100 μl/well).
5. Cell maintenance media (100 μl/well) was added to the cell monolayer and the plates incubated at 37° C. for˜24 hours
6. A crystal violet assay was performed on the plates in accordance to the Retroscreen Virology Ltd. SOP VA024-01.

Controls utilized in the toxicity assay were:
Cell only control: untreated cells. This was a negative control for toxic cytopathic effect (tCPE) and was also an indicator of cell quality.
Diluent control: cells treated with sterile double-distilled water for the specified times. This was a negative control for the test compounds and assessed any toxic effects of the diluent.
Cell and PBS control: untreated cells washed four times with PBS and incubated with cell maintenance media. This was a negative control for the washing steps, which involved a total of four washes with PBS.
   Virucidal assay
1. Cells (100 μl/well) at 1×10⁵ cells/ml were seeded onto 96-well plates and incubated at 37° C. for ˜24 hours.
2. The cell maintenance media on the plates was removed and the cell monolayer washed twice with PBS (100 μl/well).
3. Cell infection media (100 μl/well) was added to the plates.
4. Diluted virus (200 μl) of 1/2000 viral stock solution was added to each test compound (200 μl) and left to incubate at room temperature for the various incubation times specified.
5. The reaction was terminated by the addition of cell infection media (3.6 ml), which diluted the reaction 10-fold.
6. The termination mixture was centrifuged (4000 rpm for 10 minutes) and the supernatant harvested.
7. The cell infection media in wells B4-B11 of the 96-well plate was removed. The supernatant (111 μl/well) was added to wells B8-B11, and the cell only control was added to wells B4-B7. Both were plated in quadruplicate.
8. The plates were incubated at 37° C. and 5% CO₂ for 2 days.
9. On day 2 post-infection, the plates were scored for viral cytopathic effect (vCPE) and a hemagglutination (HA) assay was performed as per Retroscreen Virology Ltd. SOP VA018-02.

Controls utilized in the virucidal assay were:
Cell only control: cells not infected with virus. This is a negative control for vCPE and is also an indicator of cell quality.
Virus only control: cells infected with a 1/2000 dilution of the virus stock.
   This was a positive control for vCPE.
Diluent control: cells infected with virus that was pre-treated with sterile double-distilled water for the specified times. This was a negative control for the test compounds and assessed any antiviral effects of the diluent.
Spun virus control: cells infected with virus that was centrifuged at 4000 rpm for 10 minutes. This was a negative control for the centrifugation step and assessed whether centrifugation affected viral titer.
Antiviral control: cells infected with virus pre-treated with citrate buffer at pH3.5. This was a positive control for the test compounds.
For the virucidal assay only, the test compounds were prepared at double the concentrations than those described above. This is due to the 2-fold dilution they underwent when they were mixed with the virus.

The virucidal results demonstrate that a time-response was exhibited by R-0088 at the 0.01% w/v concentration only. At this concentration, the reductions in the Influenza A/Panama/2007/99 virus titer by R-0088 were only significant for the 10 and 30 minute incubation times. R-0089 and R-0090 did not demonstrate significant reductions in the Influenza A/Panama/2007/99 virus titer.

Thus, at the highest test concentration (0.01% w/v), R-0088 exhibited a significant reduction in the Influenza A/Panama/2007/99 virus titer at the 10 and 30 minute incubation times. R-0089 and R-0090 did not exhibit significant reductions in the Influenza A/Panama/2007/99 virus titer for any of the variables tested.

### Example 6

### Antiviral Activity of Additional Test Compounds Against Influenza A

This study was performed to determine whether additional test compounds have virucidal efficacy against an epidemic strain of Influenza A virus and to assess the cytotoxic potential of these test compounds on Madin-Darby canine kidney cells (MDCK) cells. Three different compositions of bentonite clay were studied (R-100, R-101, and R-102) to evaluate their adsorption and antiviral efficacy against an Influenza A/Panama/2007/99 (H3N2) virus.

Test compositions composed of various mineral clays (as listed below) were prepared.
R-100—Crude sodium bentonite clay.
R-101—Sodium bentonite clay having non-smectite impurities removed (as in U.S. Pat. No. 6,050,509, but without the ion exchange steps).
R-102—Purified sodium bentonite clay, purified in accordance with U.S. Pat. No. 6,050,509.
C8166 growth media (negative control)
20% Ethanol/PBS (positive control)

Each bentonite clay mixture was studied at three different concentrations (0.01% w/v, 0.001% w/v, and 0.0001% w/v prepared in sterile double-distilled water) and at three different incubation times (10 minutes, 30 minutes, and 60 minutes).

The cells of the toxicity controls were incubated with cell maintenance media, whereas the cells of the virucidal controls were incubated with cell infection media. The stock titer of Influenza A/Panama/2007/99 virus was 7.4 log₁₀ TCID₅₀/ml. Before use in the virucidal assay, the stock virus was diluted 2000-fold in infection media. It was then diluted a further 2-fold when it was mixed with the test compounds, a further 10-fold when it was mixed with the anti-viral control. The protocols for the toxicity assay and the virucidal assay are set out below.

Toxicity assay

The toxicity assay was performed as set out in Example 2 except for one modification; in step (1) of the assay, cells were seeded at (100 μl/well) at 5×104 cells/ml.

Controls utilized in the toxicity assay were:
Cell only control: untreated cells. This was a negative control for toxic cytopathic effect (tCPE) and was also an indicator of cell quality.
Diluent control: cells treated with sterile double-distilled water for the specified times. This was a negative control for the test compounds and assessed any toxic effects of the diluent.
PBS wash control: untreated cells washed four times with PBS and incubated with cell maintenance media. This was a negative control for the washing steps, which involved a total of four washes with PBS.
   Virucidal assay
1. Cells (100 μl/well) at 5×10⁴ cells/ml or 7×10⁴ cells/ml were seeded onto 96-well plates and incubated at 37° C. for ˜24 hours.
2. The cell maintenance media on the plates was removed and the cell monolayer washed twice with PBS (100 μl/well).
3. Cell infection media (100 μl/well) was added to the plates.
4. Diluted virus (200 μl) of 1/2000 viral stock solution was added to each test compound (200 μl) and left to incubate at room temperature for the various times specified. (For the antiviral control, 40 μl of the diluted virus was added to 360 μl of citrate buffer.)
5. The reaction was terminated by the addition of cell infection media (3.6 ml), which diluted the reaction 10-fold.
6. The termination mixture was centrifuged (4000 rpm for 10 minutes) and the supernatant harvested.
7. The cell infection media in wells B4-B11 of the 96-well plate was removed. The supernatant (111 μl/well) was added to wells B8-B11, and the virus only control (1/2000 viral stock solution) was added to wells B4-B7. Both were plated in quadruplicate.
8. The plates were incubated at 37° C. and 5% CO2 for 2-3 days.
9. On day 2 or 3 post-infection, the plates were scored for vCPE and an HA assay was performed as per Retroscreen Virology Ltd. SOP VA018-02.

Controls utilized in the virucidal assay were:
Cell only control: cells not infected with virus. This is a negative control for vCPE and is also an indicator of cell quality.
Virus only control: cells infected with a 1/2000 dilution of the virus stock. This was a positive control for vCPE.
Diluent control: cells infected with virus that was pre-treated with sterile double-distilled water for the specified times. This was a negative control for the test compounds and assessed any antiviral effects of the diluent.
Antiviral control: cells infected with virus pre-treated with citrate buffer at pH3.5. This was a positive control for the test compounds.

For the vinicidal assay only, the test compounds were prepared at double the concentrations than those described above. This is due to the 2-fold dilution they underwent when they were mixed with the virus.

R-100, R-101, and R-102 all exhibited time-dependent response toxicity against MDCK cells. R-100, R-101, and R-102 all exhibited a dose-response activity against Influenza A/Panama/2007/99. All the test concentrations of each test compound exhibited time-dependent response activity against Influenza A/Panama/2007/99. Only the highest test concentration (0.01% w/v) of each test compound exhibited significant reductions in virus titer at every incubation time tested.

The toxicity data generated shows that a time-response, and not a dose-response, was exhibited by the test compounds. This confirms earlier research that the incubation time rather than the test compound concentration is the determining factor of toxicity. It was also observed that the survivability of MDCK cells was also affected by the diluent control, as the values generated for the diluent control and the test compounds were similar.

After examining all the data examining toxicity, viral reduction, and therapeutic index, it was determined that there was a difference between the test compounds, but this difference was only marked when at a concentration of 0.01% w/v. As there was a difference between the toxicity of the test compounds, this suggested that the diluent, which remained consistent between the test compounds, has minimal toxicity. Toxicity and reductions in viral titer increased between R-100, R-101, and R-102 respectively. However small changes in percent toxicity for the 0.01% w/v concentration for all the test compounds had considerable impacts on the therapeutic index values.

In summary, R-102 at the highest concentration (0.01% w/v) affected the greatest reduction in viral titer with the highest therapeutic index.

### Virisorb Applications and Additional Examples

| | Example | Method of producing | Examples |
|---|---|---|---|
| 7 | Tissue & Towels | A gel comprised of water, the virucidal | The virucidal agent was a protonated |
| | | agent, and other ingredients known to the | montmorillonite added to deionized water |
| | | art is applied to the substrate that can be | in a concentration of 1% by weight. |
| | | composed of synthetic or natural fibers by | Between 0.0001% and 5% of the virucidal |
| | | either spraying, roll coating, dipping into a | agent, preferably 3% to 5%, is |
| | | trough containing the above described gel. | contemplated although higher |
| | | The final composition would contain the | percentages are useful. The slurry was |
| | | virucidal agent dispersed throughout. | uniformly sprayed onto a disposable |
| | | | “Bounty” towel in an amount equal to 5 |
| | | | times the weight of the original towel. The |
| | | | saturated towel was dried at 60° C. for 1 |
| | | | hour at which time it was determined that |
| | | | the water has been removed and the |
| | | | virucidal agent (protonated |
| | | | montmorillonite) remains on the towel. |
| | | | Other components that could be added to |
| | | | the gel include antimicrobials and |
| | | | disinfectants. |
| 8 | Masks and | The article of the above example is dried | The virucidal agent was a copper |
| | Disposable | by any number of methods well known to | exchanged montmorillonite added to |
| | Medical gowns. | the art. After drying the resultant fabric | deionized water in a concentration of 1% |
| | Air filters, | can be combined with another nonwoven | by weight. Between 0.0001% and 5%, |
| | | material using common laminating | preferably 3% to 5% of the virucidal agent |
| | | techniques. The outer layer of such a | is contemplated although higher |
| | | composition would contain the virucidal | percentages are useful. The slurry was |
| | | composition and can be further converted | uniformly sprayed onto a disposable 3M |
| | | into a disposal mask, air filter, medical | dust mask in an amount equal to 10 times |
| | | gown, bandage, bed pad, and various | the weight of the original mask. The |
| | | articles of clothing. | saturated mask was dried at 80° C. for 1 |
| | | | hour at which time it was determined that |
| | | | the water has been removed and the |
| | | | virucidal agent (copper montmorillonite) |
| | | | remained on the towel. Other |
| | | | components that could be added to the |
| | | | gel include antimicrobials, and |
| | | | disinfectants. |
| 9 | Wall paper | The article of the above composition is | |
| | | dried by any number of methods. The | |
| | | composition is combined with another | |
| | | fabric or paper through commonly known | |
| | | laminating methods. The second material | |
| | | containing, on one of its sides, an | |
| | | adhesive that can be activated by any | |
| | | number of solvents. Said composition can | |
| | | then be used in clean room environments | |
| | | as a virus resistant wall covering. | |
| 10 | Wet Wipes | A gel comprised of water, the virucidal | The virucidal agent was a silver |
| | | agent, and other ingredients useful for | exchanged montmorillonite added to |
| | | cleaning surfaces is applied to a substrate | deionized water in a concentration of 1% |
| | | composed of either synthetic or natural | by weight. Between 0.0001% and 5%, |
| | | fibers by either spraying, coating by roller | preferably 1% to 5% of the virucidal agent, |
| | | or slot die, dipping into a trough containing | is contemplated, although higher |
| | | the gel, gravure or flexographic printing, | percentages are useful. The slurry was |
| | | inkjet printing, and other means known to | 1% clay uniformly sprayed onto a |
| | | the art. Said composition is further | nonwoven substrate in an amount equal to |
| | | converted by cutting and folding into a wet | 20 times the weight of the original |
| | | wipe. The wet wipe can then be used to | nonwoven substrate. Other components |
| | | clean various surfaces depositing the gel | that could be added to the gel include |
| | | from the substrate to the surface, | antimicrobials, and disinfectants. |
| | | including human skin, animal skin, wood, | |
| | | metal, and plastic surfaces in hospitals, | |
| | | homes, and office buildings, schools, and | |
| | | similar institutions. Wet wipes could also | |
| | | be used to clean and sanitize medical | |
| | | instruments, such as surgical tools, bed | |
| | | pans, and trays. All surfaces treated with | |
| | | the wet wipe would have the virucidal | |
| | | properties of the virucidal agent. | |
| 11 | Paints for clean | A liquid composition comprised of water, | The virucidal agent in an amount of at |
| | rooms | the virucidal agent and other ingredients | least 0.01% by weight, e.g., 0.01 to 10%, |
| | | known to be useful in paint and coating | is added to a formula containing 10-40% |
| | | applications including but not limited to | pigments, 30-55% water, one or more |
| | | pigments, surfactants, emulsifiers, | latex compounds, such as, vinyl-acetate, |
| | | solvents such as binders composed of | vinyl-acrylate, acrylate, vinyl-acrylate - |
| | | vinyl acetate, vinyl acrylate, acrylate, | ethylene, and vinyl-ethylene, urethane- |
| | | urethane or combinations thereof; | acrylate emulsions in the amount ranging |
| | | epoxies, polyesters, and other setting | from 5-25%.. The above composition |
| | | compounds as well as solvents useful for | can be applied to walls, floors, and other |
| | | enabling their compounding, are applied | surfaces. |
| | | to walls, floors, counter-tops with a roller, | |
| | | brush, or by air or airless spraying | |
| | | methods. The composition upon | |
| | | application will inactivate any viruses on | |
| | | the surfaces it ahs been applied to. | |
| | | Further after application, the composition | |
| | | will retain the ability to further inactivate | |
| | | any viruses that come in contact with the | |
| | | surfaces in the future. | |
| 12 | Laundry additives | The virucidal agent is combined with | |
| | | zeolites, surfactants, and other ingredients | |
| | | commonly used in a laundry detergent. | |
| | | The composition can then be used as a | |
| | | virucidal agent for cleaning washable | |
| | | materials. | |
| 13 | Absorbent mat | A plurality of fibers are combined with the | |
| | with antimicrobial | virucidal agent and alternatively absorbent | |
| | and virucidal | polymers, antimicrobials and anti- | |
| | capability | bacterials. Additional agents to reduce | |
| | | odor may also be included. The final mat | |
| | | is then capable of absorbing fluids and | |
| | | rendering them non infectious | |
| | | alternatively, the mat can be placed over | |
| | | spills of infectious materials and used to | |
| | | absorb these fluids and render them | |
| | | noninfectious. | |
| 14 | Carpet cleaners | The virucidal agent is combined with talc, | The sodium montmorillonite virucidal |
| | and upholstery | sodium bicarbonate, surfactants, | agent was combined in a weight amount |
| | | fragrances and other ingredients | of 70% with 15% talc and 15% sodium |
| | | commonly used in powdered carpet and | bicarbonate. The mixture was a light |
| | | upholstery cleaners. The composition can | colored free flowing powder and can be |
| | | then be used as a virucidal agent by | sprinkled on carpet or upholstery where it |
| | | pouring or sprinkling on the carpet and | will interact with any virus present, easily |
| | | upholstery where it will interact with the | removes the carpet cleaner and bound |
| | | virus and can be subsequently vacuumed | virus molecules as determined by removal |
| | | up. | of the light colored material. |
| 15 | Condom Coating | A gel comprised of water, the virucidal | To a coating solution comprised of |
| | | agent, anti- agent and other ingredients | glycerine, polyethylene glycol or a mixture |
| | | known to the art is applied to the condom | of water, a humectant and a thickener |
| | | prior to packaging. The final composition | such as hydroxylpropyl cellulose is added |
| | | would contain the virucidal agent | the virucidal agent in a concentration of at |
| | | dispersed throughout. In event of condom | least 0.001% up to 30 wt. %. The coating |
| | | failure, the virucidal agent would interact | solution is then placed on the condom to |
| | | with virus released by the male or virus | completely lubricate the surface. The |
| | | already present in the partner to prevent | mixture may also include anti-spermicidal |
| | | infection of either partner. | agents such as Nonoxynol-9. |
| 16 | Vaginal Gel | A gel, crème, or body heat dissolving | The virucidal agent is incorporated in a |
| | | tablet or suppository comprised of water, | water-based formulation that contains |
| | | the virucidal agent, and other ingredients | greater than 0.001% of the |
| | | known to the art is inserted into the vagina | Montmorillonite and includes thickeners |
| | | prior to sexual activity. The final | for the water, such as xanthane gum or |
| | | composition would contain the virucidal | Carbopol along with humectants like |
| | | agent dispersed throughout. The virucidal | glycerine and propylene glycol. |
| | | agent would interact with virus released by | Alternatively, the virucidal agent could be |
| | | the male or virus already present in the | dispersed in a non-aqueous vehicle like |
| | | partner to prevent infection of either | glycerine, propylene glycol or polyethylene |
| | | partner. The product could also be used | glycol. |
| | | in a douche format to cleanse vaginal area | |
| | | after sexual intercourse and deactivate | |
| | | viruses. | |
| 17 | Hand Sanitizer | A hand sanitizer gel comprised of water, | The formula contained from about 40% to |
| | | the virucidal agent, anti-microbial agent | about 70% by weight ethyl alcohol, 30- |
| | | and other ingredients known to the art is | 60% water, glycerin, Carbomer and 1% by |
| | | applied to the hand to improve sanitation. | weight of the sodium montmorillonite |
| | | The final composition would contain the | virucidal agent. The virucidal agent can |
| | | virucidal agent dispersed throughout. | be in an amount of 0.001% to 15% by |
| | | Viricudal agent would inactivate virus | weight. The formula was rubbed on |
| | | present on the hands. | hands to provide for instant sanitization |
| | | | and inactivation of hand-held viruses. |
| 18 | Gastrointestinal | Virucidal agent our compounds are | |
| | Agent | ingested. In gastrointestinal tract, they | |
| | | interact with viruses and prevent infection. | |
| | | When wastes are expelled, viruses are | |
| | | retained on our materials and prevented | |
| | | from causing secondary infections. | |
| 19 | Nasal Lubricant | A solution/spray of the virucidal agent is | A gel comprised of water and the sodium |
| | | placed into nasal passages where it coats | Montmorillonite agent in a weight |
| | | nasal cells. When a virus contacts the | percentage from 0.00001% to 15%, more |
| | | virucidal agent, it is inactivated and | preferably 1-7%, is combined with non- |
| | | prevents infection | swelling sodium polyacrylate, know by the |
| | | | trade name CARBOPOL ®. Said gel is |
| | | | placed in a squeeze bottle with a nozzle in |
| | | | its top capable of being safely inserted |
| | | | into the nasal cavity. The gel is sprayed |
| | | | into the nasal passages by squeezing the |
| | | | bottle. The above gel may also contain |
| | | | one or more of the following materials - |
| | | | decongestants, such as Phenylephrine |
| | | | Hydrochloride, and other cold relief, |
| | | | menthol, camphor, sodium chloride, |
| | | | thimerosol and other ingredients known in |
| | | | the art. |
| 20 | Dialysis Filter | The virucidal agent is placed in a filter | The virucidal agent is present in the filter |
| | | canister and blood product is pumped | either as a 100% pure media or diluted |
| | | through the filter. The virucidal agent | 10-90% with a porosity aid. The blood |
| | | interacts with the virus present in the | product is circulated through the filter |
| | | blood product influent to reduce and | where the virucidal agent interacts with |
| | | maintain the viral count at an acceptable | the virus present in the blood product to |
| | | level. The acceptable level is dependent | maintain the viral count below an |
| | | on the virus desired to be removed. | acceptable level. It is envisioned that a |
| | | | protonated silver exchanged |
| | | | montmorillonite would be an effective |
| | | | virucidal product for dialysis filters. |
| 21 | Spill Containment | The virucidal agent is combined with other | The sodium montmorillonite virucidal |
| | | absorbent and adsorbent materials such | agent was combined in a weight |
| | | as vermiculite, sodium bentonite, oil | percentage of 50% with 30% oil adsorbent |
| | | adsorbents, polyacrylate superabsorbent | and 20% polyacrylate superabsorbent |
| | | polymers, and surfactants. In the event of | polymer. The mixture was a free flowing |
| | | a spill of a virus containing solution in a | powder and can be poured on an organic |
| | | medical associated laboratory, the | or aqueous based spill where it will |
| | | virucidal agent containing spill | interact with any virus present. |
| | | containment mixture is poured on the spill | Vacuuming easily removes the spill |
| | | area and the liquid as well as the virus is | containment agent as determined by |
| | | contained and cleaned up by shovel, or | removal of the brown colored material. |
| | | sweeping. | |

### Gel and Stick Compositions

| | Example | Method of producing |
|---|---|---|
| 22 | Vaginal Inserts/STD's | A liquid composition comprised of water, the virucidal agent, and other |
| | | cosmetically and pharmaceutically acceptable ingredients such as |
| | | glycerin, sorbitol, ethyl alcohol, thickeners such as xanthan gum, and the |
| | | like, surfactants, such as lauryl sulfate, and the like. The composition can |
| | | then be used as a gel for applying on male genetalia, vaginal inserts and |
| | | nasal sprays. |
| 23 | Hand sanitizers | The composition of the above example can be combined with ethyl |
| | | alcohol, and/or other antimicrobials such as triclosan, and/or cetyl |
| | | pyridinium chloride and the like. This composition can be used as an |
| | | instant hand sanitizer with enhanced ability to inactivate viruses. |
| 24 | Nasal Gel/spray | The composition of example 22 can be inserted or sprayed into the nasal |
| | | passages |
| 25 | Cold Sore Treatment | The composition of example 22 can be applied to cold sores to aid in |
| | | reducing the duration of cold sores through inactivating the herpes virus. |
| 26 | Alternative Lip and genitalia | An anhydrous gel containing one or more of anhydrous ingredients |
| | protectant | including waxes, synthetic and natural oils, silicones, petrolatum and the |
| | | virucidal agent are mixed together. The compositing is melted and poured |
| | | into a mold, commonly used to form lip coating products. Upon cooling, |
| | | the materials are removed from the molds and can be used as lipsticks, lip |
| | | balms, vaginal inserts, and the like. |
| 27 | Emulsion | Water containing the virucidal agent, and surfactants and lipophilic |
| | | materials such as waxes, synthetic or natural oils, silicones, |
| | | hydrocarbons, and similar materials can be combined by mixing under |
| | | high shear to create an emulsion. This emulsion can be used directly on |
| | | human skin, animal skin and various surfaces as a virucidal agent. |
| | | Alternatively, the composition can be applied to substrates and dried to |
| | | create a filter, bandage and mask. In addition, the emulsion can be |
| | | applied to a substrate that is further converted into a wet wipe that can be |
| | | used to apply the virucidal composition to various surfaces. |
| 28 | Filter device for removing | The virucidal agent is placed in a cartridge that has a porous cover, or a |
| | virus from fluids | plurality of holes, that enables liquid to flow through the cartridge, but |
| | | retain the virucidal agent within it. The device can then be used to |
| | | inactivate viruses in the blood stream of animals or humans, water, and |
| | | any other liquid that may contain viruses. |
| 29 | Blood adsorbent with virus | The virucidal agent is combined with absorbent polymers and other anti |
| | inactivation | microbial or antibacterial agents, such as CPC, triclosan, and the like. |
| | | The powder is then capable of solidifying liquid and semi-solid wastes |
| | | from animals and humans and inactivating viruses present in the wastes, |
| | | eliminating the potential for spreading infectious diseases. |

### Example 30

### Antiviral Activity of Test Compounds Against Feline Calcivirus

In this study, three different compositions of bentonite clay were studied (R-400, R-401, and R-402) to evaluate their antiviral efficacy against a feline calcivirus (a surrogate for Norovirus) (ATCC VR-782).

Test substances.

R-400: purified homoionic sodium bentonite mixture, purified in accordance with U.S. Pat. No. 6,050,509

R-401: purified homoionic hydrogen (protonated) bentonite mixture

R-402: purified homoionic hydrogen (protonated) bentonite #2 mixture

Each of the test substances were dispersed in double distilled water at a concentration of 0.1% (w/v) prior to use in the following assays.

Virus and Preparation of Stock Virus. The F-9 strain of the Feline Calcivirus stock virus was obtained from the American Type Culture collection, Manassas, Va. (ATCC VR-782). Stock virus was prepared by collecting the supernatant culture fluid from infected culture cells. The cells were disrupted and cell debris removed by centrifugation at 2000 RPM for five minutes at 4° C. The supernatant was removed, aliquoted, and the high titer stock virus was stored at ≦−70° C. until the day of use. On the day of use, five aliquots of stock virus (ATS Labs Lot FC-33) were removed, thawed, combined and refrigerated until use in the assay. The stock virus culture contained 5% fetal bovine serum (FBS) as the organic soil load. The stock virus tested demonstrated cytopathic effects (CPE) typical of Feline Calcivirus on feline kidney cells.

Test Cell Cultures. Cultures of feline kidney (CRFK) cells were originally obtained from the American Type Culture collection, Manassas, VA (ATCC CCL-94). The cells were propagated, seeded into multi-well cell culture plates and maintained at 36-38° C. in a humidified atmosphere of 5-7% CO₂.

Test Medium. The test medium used in the following assays was Minimum Essential Medium (MEM), supplemented with 5% heat-inactivated fetal bovine serum (FBS), 10 μg/mL gentamicin, 100 U/mL penicillin, and 2.5 μg/mL amphotericin B.

Preparation of Test Substance. Each of R-400, R-401 and R-402 were shaken vigorously by hand for three minutes, aliquoted and utilized immediately in the following assays.

Treatment of Virus Suspension. For each exposure temperature (room temperature and 37° C.), A 4.5 mL aliquot of test substance was dispensed into separate sterile 15 mL conical tubes and mixed with a 0.5 mL aliquot of the stock virus suspension. The mixtures were vortex mixed for ten seconds and held for the remainder of the specified 30 second exposure time at room temperature (actual 24.5° C.) and at 37±1° C. (actual 37.0° C.). Immediately following the exposure time, a 0.1 mL aliquot was removed from each tube and the mixtures were titered by 10-fold serial dilution (0.1 mL+0.9 mL test medium). To decrease the test substance cytotoxicity, the first dilution was made in FBS with the remaining dilutions in test medium. Each assay was then assayed for the presence of the virus.

Treatment of Virus control: A 0.5 mL aliquot of stock Virus suspension was exposed for 30 seconds to a 4.5 mL aliquot of test medium in lieu of test substance at exposure temperatures of 24.5° C. and 37.1° C. Immediately following the exposure time, a 0.1 mL aliquot was removed from each tube and the mixtures were titred by 10-fold serial dilution (0.1 mL+0.9 mL test medium). All controls employed the FBS neutralized as described in the Treatment of Virus Suspension section. The virus control titer was used as a baseline to compare the percent and log reductions of each test parameter following exposure to the test substances.

Cytotoxicity Controls. A 4.5 mL aliquot of each test substance was mixed with a 0.5 mL aliquot of test medium containing 5% FBS in lieu of virus and treated as previously described for each exposure temperature assayed. The cytotoxicity of the cell cultures was scored at the same time as virus-test substance and virus control cultures. Cytotoxicity was graded on the basis of cell viability as determined microscopically. Cellular alterations due to toxicity were graded and reported as toxic (T) if greater than or equal to 50% of the monolayer was affected.

Neutralization Controls. Each cytotoxicity control mixture was challenged with low titer stock virus to determine the dilution(s) of test substance at which virucidal activity, if any, was retained. Dilutions that showed virucidal activity were not considered in determining reduction of the virus by the test substance.

Neutralization Assay. As described above, 0.1 mL of each test and control parameter following the exposure time period was added to FBS (0.9 mL) followed immediately by 10-fold serial dilutions in test medium to stop the action of the test substance. To determine if the neutralizer chose for the assay was effective in diminishing the virucidal activity of the test substance, low titer stock virus was added to each dilution of the test substance-neutralizer mixture. The mixtures were assayed for the presence of virus.

Infectivity Assay. The CRFK cell line, which exhibits CPE in the presence of Feline Calcivirus, was used as the indicator cell line in the infectivity assays. Cells in multiwell culture dishes were inoculated in quadruplicate with 0.1 mL of the dilutions prepared from test and control groups. Uninfected indicator cell cultures (cell controls) were inoculated with test medium alone. The cultures were incubated at 31-35° C. in a humidified atmosphere of 5-7% CO₂ in sterile disposable cell culture lab ware. The cultures were microscopically scored periodically for seven days for the absence or presence of CPE, cytotoxicity, and for viability.

Results for Test Substances R-400, R-401 and R-402. Following a 30 second exposure time at room temperature (24.5° C.), test virus infectivity was detected in the virus-test substance mixture at 7.0 log₁₀. Test substance cytotoxicity was detected at 3.5 log₁₀. The neutralization control demonstrated that the test substance was neutralized at ≦2.5 log₁₀. Taking the cytotoxicity and neutralization control results into consideration, R-400 demonstrated an 82.2% reduction (at 24.5° C.) and a ≧96.8% reduction (at 37.0° C.) in viral titer following a 30 second exposure time to the virus. The log reductions in viral titer were 0.75 log₁₀ and ≧1.5 log₁₀, respectively. R-401 demonstrated a 43.8% reduction (at 24.5° C.) and a ≧96.8% reduction (at 37.0° C.) in viral titer following a 30 second exposure time to the virus. The log reduction in viral titers was 0.25 log₁₀ and ≧1.5 log₁₀, respectively. R-0402 demonstrated a 68.4% reduction (at 24.5° C.) and a ≧96.8% reduction (at 37.0° C.) in viral titer following a 30 second exposure time to the virus. The log reductions in viral titer were 0.5 log₁₀ and ≧1.5 log₁₀, respectively.

### Example 31

### Antiviral Activity of Test Compounds Against Rotavirus

In this study, three different compositions of bentonite clay were studied (R-400, R-401, and R-402) to evaluate their adsorption and antiviral efficacy against Rotavirus.

Test substances.

R-400: purified homoionic sodium bentonite mixture, purified in accordance with U.S. Pat. No. 6,050,509

R-401: purified homoionic hydrogen (protonated) bentonite mixture

R-402: purified homoionic hydrogen (protonated) bentonite #2 mixture

Each of the test substances were dispersed in double distilled water at a concentration of 0.1% (w/v) prior to use in the following assays.

Viris and Preparation of Stock Virus. The WA strain of Rotavirus was obtained from the University of Ottawa, Ontario, Canada. Stock virus was prepared by collecting the supenatant culture fluid from infected culture cells. The cells were disrupted and cell debris removed by centrifugation at 2000 RPM for five minutes at approximately 4° C. The supernatant was removed, aliquoted, and the high titer stock virus was stored at ≦−70° C. until the day of use. On the day of use, five aliquots of stock virus (ATS Labs Lot XR-115) were removed, thawed, combined and refrigerated until use in the assay. The stock virus culture contained 5% fetal bovine serum (FBS) as the organic soil load. The stock virus tested demonstrated cytopathic effects (CPE) typical of Rotavirus on MA-104 cells.

Test Cell Cultures. Cultures of MA-104 (Rhesus monkey kidney) cells were originally obtained from Diagnostics Hybrids Inc., Athens, Ohio. The cells were propagated, seeded into multiwell cell culture plates and maintained at 36-38° C. in a humidified atmosphere of 5-7% CO₂.

Test Medium. The test medium used in the following assays was serum free Minimum Essential Medium (MEM), supplemented with 0.5 μg/mL trypsin, 2.0 mM L-glutamine, 10 μg/mL gentamicin, 100 U/mL penicillin, and 2.5 μg/mL amphotericin B.

Preparation of Test Substance. Each of R-400, R-401 and R-402 were shaken vigorously by hand for three minutes, aliquoted and utilized immediately in the following assays.

Treatment of Virus Suspension. For each exposure temperature (room temperature and 37° C.), a 4.5 mL aliquot of test substance was dispensed into separate sterile 15 mL conical tubes and mixed with a 0.5 mL aliquot of the stock virus suspension. The mixtures were vortex mixed for ten seconds and held for the remainder of the specified 30 second exposure time at room temperature (actual 24.5° C.) and at 37±1° C. (actual 38.0° C.). Immediately following the exposure time, a 0.1 mL aliquot was removed from each tube and the mixtures were titered by 10-fold serial dilution (0.1 mL+0.9 mL test medium). To decrease the test substance cytotoxicity, the first dilution was made in FBS with the remaining dilutions in test medium. Each assay was then assayed for the presence of the virus.

Treatment of Virus Control: A 0.5 mL aliquot of stock virus suspension was exposed for 30 seconds to a 4.5 mL aliquot of test medium in lieu of test substance at exposure temperatures of 24.5° C. and 38.0° C. Immediately following the exposure time, a 0.1 mL aliquot was removed from each tube and the mixtures were titered by 10-fold serial dilution (0.1 mL+0.9 mL test medium). All controls employed the FBS neutralized as described in the Treatment of Virus Suspension section. The virus control titer was used as a baseline to compare the percent and log reductions of each test parameter following exposure to the test substances.

Cytotoxicity Controls. A 4.5 mL aliquot of each test substance was mixed with a 0.5 mL aliquot of test medium containing 5% FBS in lieu of virus and treated as previously described for each exposure temperature assayed. The cytotoxicity of the cell cultures was scored at the same time as virus-test substance and virus control cultures. Cytotoxicity was graded on the basis of cell viability as determined microscopically. Cellular alterations due to toxicity were graded and reported as toxic (T) if greater than or equal to 50% of the monolayer was affected.

Neutralization Controls. Each cytotoxicity control mixture was challenged with low titer stock virus to determine the dilution(s) of test substance at which virucidal activity, if any, was retained. Dilutions that showed virucidal activity were not considered in determining reduction of the virus by the test substance.

Neutralization Assay. As described above, 0.1 mL of each test and control parameter following the exposure time period was added to FBS (0.9 mL) followed immediately by 10-fold serial dilutions in test medium to stop the action of the test substance. To determine if the neutralizer chose for the assay was effective in diminishing the virucidal activity of the test substance, low titer stock virus was added to each dilution of the test substance-neutralizer mixture. The mixtures were assayed for the presence of virus.

Infectivity Assay. The MA-104 cell line, which exhibits CPE in the presence of Rotavirus, was used as the indicator cell line in the infectivity assays. Cells in multiwell culture dishes were inoculated in quadruplicate with 0.1 mL of the dilutions prepared from test and control groups. Uninfected indicator cell cultures (cell controls) were inoculated with test medium alone. The inoculum was allowed to adsorb for 60 minutes at 36-38° C. in a humidified atmosphere of 5-7% CO₂. Following the adsorption period, a 1.0 mL aliquot of test medium was added to each well of the cell cultures, and the cultures were incubated at 36-38° C. in a humidified atmosphere of 5-7% CO₂ in sterile disposable cell culture lab ware. The cultures were microscopically scored periodically for seven days for the absence or presence of CPE, cytotoxicity, and for viability.

Infectivity Results for Test Substances R-400, R-401 and R-402. Following a 30 second exposure time at room temperature (24.5° C.), test virus infectivity was detected in the virus-test substance mixture at 7.25 log₁₀. Test substance cytotoxicity was detected at 3.5 log₁₀. The neutralization control demonstrated that the test substance was neutralized at ≦2.5 log₁₀. Taking the cytotoxicity and neutralization control results into consideration, R-400 demonstrated no reduction in viral titer following a 30 second exposure time to the virus at 24.5° C. or 38.0° C. R-401 also demonstrated no reduction in viral titer following a 30 second exposure time to the virus at 24.5° C. or 38.0° C. R-0402, however, demonstrated a 68.4% reduction (at 24.5° C.) and a ≧99.7% reduction (at 38.0° C.) in viral titer following a 30 second exposure time to the virus. The log reductions in viral titer were 0.5 log₁₀ and ≧2.5 log₁₀, respectively.

### Example 32

### Antiviral Activity of Test Compounds Against Influenza A

In this study, three different compositions of bentonite clay were studied (R-400, R-401, and R-402) to evaluate their adsorption and antiviral efficacy against Influenza A virus.

Test Substances.

R-400: purified homoionic sodium bentonite mixture, purified in accordance with U.S. Pat. No. 6,050,509

R-401: purified homoionic hydrogen (protonated) bentonite mixture

R-402: purified homoionic hydrogen (protonated) bentonite #2 mixture

Each of the test substances were dispersed in double distilled water at a concentration of 0.1% (w/v) prior to use in the following assays.

Virus and Preparation of Stock Virus. The Hong Kong strain of Influenza A virus was obtained from the American Type Culture Collection, Manassas, Va. (ATCC VR-544). Stock virus was prepared by collecting the supernatant culture fluid from infected culture cells. The cells were disrupted and cell debris removed by centrifugation at 2000 RPM for five minutes at approximately 4° C. The supernatant was removed, aliquoted, and the high titer stock virus was stored at ≦−70° C. until the day of use. On the day of use, two aliquots of stock virus (ATS Labs Lot F66) were removed, thawed, combined and refrigerated until use in the assay. The stock virus culture contained 1% fetal bovine serum (FBS) as the organic soil load. The stock virus tested demonstrated cytopathic effects (CPE) typical of Influenza virus on Rhesus monkey kidney cells.

Test Cell Cultures. Rhesus monkey kidney (RMK) cells were originally obtained from VioMed Laboratories, Inc., Minneapolis, Minn., Cell Culture Division. Culturew were maintained and used as monolayers in disposable tissue culture labware. On the day of testing, cells were observed as having proper cell integrity.

Test Medium. The test medium used in the following assays was Minimum Essential Medium (MEM), supplemented with 1% (v/v) heat-inactivated fetal bovine serum (FBS), 10 μg/mL gentamicin, 100 U/mL penicillin, and 2.5 μg/mL amphotericin B.

Preparation of Test Substance. Each of R-400, R-401 and R-402 were shaken vigorously by hand for three minutes, aliquoted and utilized immediately in the following assays.

Treatment of Virus Suspension. A 4.5 mL aliquot of test substance was dispensed into separate sterile 15 mL conical tubes and mixed with a 0.5 mL aliquot of the stock virus suspension. The mixtures were vortex mixed for ten seconds and held for the remainder of the specified 30 second exposure time at room temperature (actual 24.0° C.). Immediately following the exposure period, a 0.1 mL aliquot was removed from each tube and the mixtures were titered by 10-fold serial dilutions (0.1 mL+0.9 mL test medium). To decrease the test substance cytotoxicity, the first dilution was made in FBS with the remaining dilutions in test medium. Each dilution was then assayed for the presence of the virus.

Treatment of Virus Control: A 0.5 mL aliquot of stock virus suspension was exposed for 30 seconds to a 4.5 mL aliquot of test medium in lieu of test substance at 24.0° C. Immediately following the exposure time, a 0.1 mL aliquot was removed from each tube and the mixtures were titered by 10-fold serial dilutions (0.1 mL+0.9 mL test medium). All controls employed the FBS neutralized as described in the Treatment of Virus Suspension section. The virus control titer (7.25 log₁₀) was used as a baseline to compare the percent and log reductions of each test parameter following exposure to the test substances.

Cytotoxicity Controls. A 4.5 mL aliquot of each test substance was mixed with a 0.5 mL aliquot of test medium containing 1% FBS in lieu of virus and treated as previously described for the exposure temperature assayed. The cytotoxicity of the cell cultures was scored at the same time as virus-test substance and virus control cultures. Cytotoxicity was graded on the basis of cell viability as determined microscopically. Cellular alterations due to toxicity were graded and reported as toxic (T) if greater than or equal to 50% of the monolayer was affected.

Neutralization Controls. Each cytotoxicity control mixture was challenged with low titer stock virus to determine the dilution(s) of test substance at which virucidal activity, if any, was retained. Dilutions that showed virucidal activity were not considered in determining reduction of the virus by the test substance.

Neutralization Assay. As described above, 0.1 mL of each test and control parameter following the exposure time period was added to FBS (0.9 mL) followed immediately by 10-fold serial dilutions in test medium to stop the action of the test substance. To determine if the neutralizer chosen for the assay was effective in diminishing the virucidal activity of the test substance, low titer stock virus was added to each dilution of the test substance-neutralizer mixture. The mixtures were assayed for the presence of virus.

Infectivity Assay. The RMK cell line, which exhibits CPE in the presence of Influenza A virus, was used as the indicator cell line in the infectivity assays. Cells in multi-well culture dishes were inoculated in quadruplicate with 0.1 mL of the dilutions prepared from test and control groups. Uninfected indicator cell cultures (cell controls) were inoculated with test medium alone. The cultures were incubated at 36-38° C. in a humidified atmosphere of 5-7% CO₂ in sterile disposable cell culture lab ware. The cultures were microscopically scored periodically for seven days for the absence or presence of CPE, cytotoxicity, and for viability.

Antiviral Results for Test Substance R-400, R-401 and R-402. Following a 30 second exposure time at room temperature (24.5° C.), test virus infectivity was detected in the virus-test substance mixture (virus control) at 7.25 log₁₀.

Cytotoxicity of R-400 was detected at 3.5 log₁₀. The neutralization control demonstrated that the test substance was neutralized at ≦2.5 log₁₀. Following exposure, test virus infectivity was detected in the virus-test substance mixture at 7.75 log₁₀. Therefore, R-400 demonstrated no reduction in viral titer following a 30 second exposure time to the virus at 24.0° C.

Cytotoxicity of R-401 was detected at 2.5 log₁₀. The neutralization control demonstrated that the test substance was neutralized at ≦2.5 log₁₀. Following exposure, test virus infectivity was detected in the virus-test substance mixture at 7.0 log₁₀. Therefore, R-401 also demonstrated no reduction in viral titer following a 30 second exposure time to the virus at 24.0° C.

Cytotoxicity of R-402 was detected at 3.5 log₁₀. The neutralization control demonstrated that the test substance was neutralized at ≦2.5 log₁₀. Following exposure, test virus infectivity was detected in the virus-test substance mixture at 4.5 log₁₀. Therefore, R-402 demonstrated a 99.8% (2.75 log₁₀) reduction in viral titer following a 30 second exposure time to the virus at 24.0° C.

### Example 33

### Antiviral Activity of Test Compounds Against Influenza A

In this study, three different compositions of bentonite clay were studied (R-400, R-401, and R-402) to evaluate their adsorption and antiviral efficacy against Rhinovirus type 37.

Test substances.

R-400: purified homoionic sodium bentonite mixture, purified in accordance with U.S. Pat. No. 6,050,509

R-401: purified homoionic hydrogen (protonated) bentonite mixture

R-402: purified homoionic hydrogen (protonated) bentonite #2 mixture

Each of the test substances were dispersed in double distilled water at a concentration of 0.1% (w/v) prior to use in the following assays.

Virus and Preparation of Stock Virus. The 151-1 strain of Rhinovirus type 37 was obtained from the American Type Culture Collection, Manassas, Va. (ATCC VR-51147). Stock virus was prepared by collecting the supernatant culture fluid from infected culture cells. The cells were disrupted and cell debris removed by centrifugation at 2000 RPM for five minutes at approximately 4° C. The supernatant was removed, aliquoted, and the high titer stock virus was stored at ≦−70° C. until the day of use. On the day of use, two aliquots of stock virus (ATS Labs LotR37-104) were removed, thawed, combined and refrigerated until use in the assay. The stock virus culture contained 1% fetal bovine serum (FBS) as the organic soil load. The stock virus tested demonstrated cytopathic effects (CPE) typical of Rhinovirus on human embryonic lung cells.

Test Cell Cultures. Cultures of human embryonic lung (MRC-5) cells were originally obtained from American Type Culture Collection, Manassas, Va. (ATCC CCL-171). The cells were propagated, seeded into multi-well cell culture plates and maintained at 36-38° C. in a humidified atmosphere of 5-7% CO₂.

Test Medium. The test medium used in the following assays was Minimum Essential Medium (MEM), supplemented with 10% (v/v) heat-inactivated fetal bovine serum (FBS), 10 μg/mL gentamicin, 100 U/mL penicillin, and 2.5 μg/mL amphotericin B.

Preparation of Test Substance. Each of R-400, R-401 and R-402 were shaken vigorously by hand for three minutes, aliquoted and utilized immediately in the following assays.

Treatment of Virus Suspension. A 4.5 mL aliquot of test substance was dispensed into a separate sterile 15 mL conical tube and mixed with a 0.5 mL aliquot of the stock virus suspension. The mixtures were vortex mixed for ten seconds and held for the remainder of the specified 30 second exposure time at room temperature (actual 24.0° C.). Immediately following the exposure period, a 0.1 mL aliquot was removed from each tube and the mixture was titered by 10-fold serial dilutions (0.1 mL+0.9 mL test medium). To decrease the test substance cytotoxicity, the first dilution was made in FBS with the remaining dilutions in test medium. Each dilution was then assayed for the presence of the virus.

Treatment of Virus Control: A 0.5 mL aliquot of stock virus suspension was exposed for 30 seconds to a 4.5 mL aliquot of test medium in lieu of test substance at 24.0° C. Immediately following the exposure time, a 0.1 mL aliquot was removed from each tube and the mixtures were titered by 10-fold serial dilutions (0.1 mL+0.9 mL test medium). All controls employed the FBS neutralizer as described in the Treatment of Virus Suspension section. The virus control titer (5.0 log₁₀) was used as a baseline to compare the log reduction of each test parameter following exposure to the test substances.

Cytotoxicity Controls. A 4.5 mL aliquot of each test substance was mixed with a 0.5 mL aliquot of test medium containing 1% FBS in lieu of virus and treated as previously described for the exposure temperature assayed. The cytotoxicity of the cell cultures was scored at the same time as virus-test substance and virus control cultures. Cytotoxicity was graded on the basis of cell viability as determined microscopically. Cellular alterations due to toxicity were graded and reported as toxic (T) if greater than or equal to 50% of the monolayer was affected.

Neutralization Controls. Each cytotoxicity control mixture was challenged with low titer stock virus to determine the dilution(s) of test substance at which virucidal activity, if any, was retained. Dilutions that showed virucidal activity were not considered in determining reduction of the virus by the test substance.

Neutralization Assay. As described above, 0.1 mL of each test and control parameter following the exposure time period was added to FBS (0.9 mL) followed immediately by 10-fold serial dilutions in test medium to stop the action of the test substance. To determine if the neutralizer chosen for the assay was effective in diminishing the virucidal activity of the test substance, low titer stock virus was added to each dilution of the test substance-neutralizer mixture. The mixtures were assayed for the presence of virus.

Infectivity Assay. The MRC-5 cell line, which exhibits CPE in the presence of Rhinovirus type 37, was used as the indicator cell line in the infectivity assays. Cells in multi-well culture dishes were inoculated in quadruplicate with 0.1 mL of the dilutions prepared from test and control groups. Uninfected indicator cell cultures (cell controls) were inoculated with test medium alone. The cultures were incubated at 36-38° C. in a humidified atmosphere of 5-7% CO₂ in sterile disposable cell culture lab ware. The cultures were microscopically scored periodically for seven days for the absence or presence of CPE, cytotoxicity, and for viability.

Antiviral Results for Test Substance R-400, R-401 and R-402. Following a 30 second exposure time at room temperature (24.5° C.), test virus infectivity was detected in the virus-test substance mixture (virus control) at 5.0 log₁₀.

Cytotoxicity of R-400 was detected at 2.5 log₁₀. The neutralization control demonstrated that the test substance was neutralized at ≦2.5 log₁₀. Following exposure, test virus infectivity was detected in the virus-test substance mixture at 5.0 log₁₀. Therefore, R-400 demonstrated 110 reduction in viral titer following a 30 second exposure time to the virus at 24.0° C.

Cytotoxicity of R-401 was detected at 2.5 log₁₀. The neutralization control demonstrated that the test substance was neutralized at ≦2.5 log₁₀. Following exposure, test virus infectivity was detected in the virus-test substance mixture at 5.0 log₁₀. Therefore, R-401 also demonstrated no reduction in viral titer following a 30 second exposure time to the virus at 24.0° C.

Cytotoxicity of R-402 was detected at 3.5 log₁₀. The neutralization control demonstrated that the test substance was neutralized at ≦2.5 log₁₀. Following exposure, test virus infectivity was not detected in the virus-test substance mixture (≦2.5 log₁₀). Therefore, R-402 demonstrated a ≧99.7% (≦2.5 log₁₀) reduction in viral titer following a 30 second exposure time to the virus at 24.0° C.

### Example 34

### Virucidal Efficacy of a Disinfectant Against Influenza A for Use on Inanimate Surfaces

In this study, three different compositions of bentonite clay were studied (R-400, R-401, and R-402) to evaluate their virucidal efficacy against Influenza A virus.

Test Substances.

R-400: purified homoionic sodium bentonite mixture, purified in accordance with U.S. Pat. No. 6,050,509

R-401: purified homoionic hydrogen (protonated) bentonite mixture

R-402: purified homoionic hydrogen (protonated) bentonite #2 mixture

Each of the test substances were dispersed in double distilled water at a concentration of 0.1% (w/v) prior to use in the following assays.

Virus and Preparation of Stock Virus. The Hong Kong strain of Influenza A virus was obtained from the American Type Culture Collection, Manassas, Va. (ATCC VR-544). Stock virus was prepared by collecting the supernatant culture fluid from infected culture cells. The cells were disrupted and cell debris removed by centrifugation at 2000 RPM for five minutes at approximately 4° C. The supernatant was removed, aliquoted, and the high titer stock virus was stored at ≦−70° C. until the day of use. On the day of use, two aliquots of stock virus (ATS Labs Lot F66) were removed, thawed, combined and refrigerated until use in the assay. The stock virus culture contained 1% fetal bovine serum (FBS) as the organic soil load. The stock virus tested demonstrated cytopathic effects (CPE) typical of Influenza virus on Rhesus monkey kidney cells.

Test Cell Cultures. Rhesus monkey kidney (RMK) cells were originally obtained from VioMed Laboratories, Inc., Minneapolis, Minn., Cell Culture Division. Cultures were maintained and used as monolayers in disposable tissue culture lab ware. On the day of testing, cells were observed as having proper cell integrity.

Test Medium. The test medium used in the following assays was Minimum Essential Medium (MEM), supplemented with 1% (v/v) heat-inactivated fetal bovine serum (FBS), 10 μg/mL gentamicin, 100 U/mL penicillin, and 2.5 μg/mL amphotericin B.

Preparation of Test Substance. Each of R-400, R-401 and R-402 were shaken vigorously by hand for three minutes, aliquoted and utilized immediately in the following assays.

Preparation of Virus Films. Films of virus were prepared by spreading 0.2 mL of virus inoculum uniformly over the bottoms of four separate 100×15 mm sterile glass petri dishes. The virus films were dried at 19° C. in a relatively humidity of 53% until visibly dry (approximately 25 minutes).

Sephadex Gel Filtration. To reduce the cytotoxic level of the virus-test substance mixture prior to assay of virus and/or to reduce the virucidal level of the test substance, virus was separated from test substance by filtration through Sephadex gel. Columns of Sephadex LH-20-100 were equilibrated with PBS containing 1% albumin and centrifuged or three minutes to clear the void volume.

Treatment of Virus Films with Test Substance. For each test substance, separate dried virus films were exposed to 2.0 mL of the use dilution for one minute at room temperature (20.0° C.). The virus films were completely covered with the test substance. Following the exposure time, the plates were scraped with a cell scraper to re-suspend the contents of the plate and the virus-test substance mixture was immediately passed through a Sephadex column utilizing a syringe plunger in order to detoxify the mixture. The filtrate (10⁻¹ dilution) was then titered by 10-fold serial dilution and assayed for infectivity.

Treatment of Virus Control Films. A virus film was prepared as described above. The control film was exposed to 2.0 mL of test medium for one minute at room temperature (20.0° C.). The virus was then scraped and passed through a Sephadex column as described above. The filtrate (10⁻¹ dilution) was then titered by 10-fold serial dilution and assayed for infectivity.

Cytotoxicity Assay. A 2.0 mL aliquot of the use dilution of each test substance was filtered through a Sephadex column and the filtrate was diluted serially in medium and inoculated into RMK cell cultures. Cytotoxicity of the RMK cell cultures was scored at the same time as the virus-test substance and virus control cultures.

Assay on Non-Virucidal Level of Test Substance. Each dilution of the Sephadex-filtered test substance was mixed with an aliquot of low titer stock virus, and the resulting mixtures of dilutions were assayed for infectivity in order to determine the dilution(s) of test substance at which virucidal activity, if any, was retained. Dilutions that showed virucidal activity were not considered in determining the reduction in infectivity by the test substance.

Infectivity Assay. The RMK cell line, which exhibits CPE in the presence of Influenza A virus, was used as the indicator cell line in the infectivity assays. Cells in multi-well culture dishes were inoculated in quadruplicate with 0.1 mL of the dilutions prepared from test and control groups. Uninfected indicator cell cultures (cell controls) were inoculated with test medium alone. The cultures were incubated at 36-38° C. in a humidified atmosphere of 5-7% CO₂ in sterile disposable cell culture lab ware. The cultures were microscopically scored periodically for seven days for the absence or presence of CPE, cytotoxicity, and for viability.

Virucidal Results for Test Substance R-400, R-401 and R-402. Results of tests with R-0400, R-0401 and R-0402 exposed to Influenza A virus in the presence of a 5% fetal bovine serum soil load at 20.0° C. for one minute are set forth below. All cell controls were negative for test virus infectivity. The titer of the dried virus control was 5.75 log₁₀.

Following exposure, test virus infectivity was detected in the virus-test substance mixture at 2.5 log₁₀ for test substance R-0400, 5.25 log1₀ for test substance R-0401 and 1.0 log₁₀ for test substance R-0402.

Test substance cytotoxicity was observed in test substance R-0400 at 1.5 log₁₀ and in test substance R-0401 at 2.5 log10. Test substance cytotoxicity was not observed in test substance R-0402 at any dilution tested (≦0.5 log₁₀).

The neutralization control (non-virucidal level of the test substance) indicates that the test substance R-0400 was neutralized at ≦1.5 log₁₀, test substance R-0401 was neutralized at ≦2.5 log₁₀ and test substance R-0402 was neutralized at <0.5 log₁₀.

Taking the cytotoxicity and neutralization control results into consideration, the reduction in viral titer was 3.25 log₁₀ for test substance R-0400, 0.5 log₁₀ for test substance R-0401 and 4.75 log₁₀ for test substance R-0402. Therefore, none of the test substances demonstrated complete inactivation of Influenza A virus following a one minute exposure time at 20.0° C. as required by the U.S. Environmental Protection Agency (EPA) for virucidal label claims.

### Example 35

### Virucidal Efficacy of a Disinfectant Against Rhinovirus Type 37 for Use on Inanimate Surfaces

In this study, three different compositions of bentonite clay were studied (R-400, R-401, and R-402) to evaluate their virucidal efficacy against Rhinovirus type 37.

Test Substances.

R-400: purified homoionic sodium bentonite mixture, purified in accordance with U.S. Pat. No. 6,050,509

R-401: purified homoionic hydrogen (protonated) bentonite mixture

R-402: purified homoionic hydrogen (protonated) bentonite #2 mixture

Each of the test substances were dispersed in double distilled water at a concentration of 0.1% (w/v) prior to use in the following assays.

Virus and Preparation of Stock Virus. The 151-1 strain of Rhinovirus type 37 was obtained from the American Type Culture Collection, Manassas, Va. (ATCC VR-1147). Stock virus was prepared by collecting the supenatant culture fluid from infected culture cells. The cells were disrupted and cell debris removed by centrifugation at 2000 RPM for five minutes at approximately 4° C. The supernatant was removed, aliquoted, and the high titer stock virus was stored at ≦−70° C. until the day of use. On the day of use, two aliquots of stock virus (ATS Labs Lot R37-105) were removed, thawed, combined and refrigerated until use in the assay. The stock virus culture contained 1% fetal bovine serum (FBS) as the organic soil load. The stock virus tested demonstrated cytopathic effects (CPE) typical of Rhinovirus on human embryonic lung cells.

Test Cell Cultures. Cultures of human embryonic lung (MRC-5) cells were originally obtained from American Type Culture Collection, Manassas, Va. (ATCC CCL-171). The cells were propagated, seeded into multi-well cell culture plates and maintained at 36-38° C. in a humidified atmosphere of 5-7% CO₂.

Test Medium. The test medium used in the following assays was Minimum Essential Medium (MEM), supplemented with 10% (v/v) heat-inactivated fetal bovine serum (FBS), 10 μg/mL gentamicin, 100 U/mL penicillin, and 2.5 μg/mL amphotericin B.

Preparation of Test Substance. Each of R-400, R-401 and R-402 were shaken vigorously by hand for three minutes, aliquoted and utilized immediately in the following assays.

Preparation of Virus Films. Films of virus were prepared by spreading 0.2 mL of virus inoculum uniformly over the bottoms of four separate 100×15 mm sterile glass petri dishes. The virus films were dried at 18.5° C. in a relatively humidity of 56% until visibly dry (approximately 20 minutes).

Sephadex Gel Filtration. To reduce the cytotoxic level of the virus-test substance mixture prior to assay of virus and/or to reduce the virucidal level of the test substance, virus was separated from test substance by filtration through Sephadex gel. Columns of Sephadex LH-20-100 were equilibrated with PBS containing 1% albumin and centrifuged or three minutes to clear the void volume.

Treatment of Virus Films with Test Substance. For each test substance, separate dried virus films were exposed to 2.0 mL of the use dilution for one minute at room temperature (19.5° C.). The virus films were completely covered with the test substance. Following the exposure time, the plates were scraped with a cell scraper to re-suspend the contents of the plate and the virus-test substance mixture was immediately passed through a Sephadex column utilizing a syringe plunger in order to detoxify the mixture. The filtrate (10⁻¹ dilution) was then titered by 10-fold serial dilution and assayed for infectivity.

Treatment of Virus Control Films. A virus film was prepared as described above. The control film was exposed to 2.0 mL of test medium for one minute at room temperature (20.0° C.). The virus was then scraped and passed through a Sephadex column as described above. The filtrate (10-1 dilution) was then titered by 10-fold serial dilution and assayed for infectivity.

Cytotoxicity Assay. A 2.0 mL aliquot of the use dilution of each test substance was filtered through a Sephadex column and the filtrate was diluted serially in medium and inoculated into MRC-5 cell cultures. Cytotoxicity of the RMK cell cultures was scored at the same time as the virus-test substance and virus control cultures.

Assay on Non-Virucidal Level of Test Substance. Each dilution of the Sephadex-filtered test substance was mixed with an aliquot of low titer stock virus, and the resulting mixtures of dilutions were assayed for infectivity in order to determine the dilution(s) of test substance at which virucidal activity, if any, was retained. Dilutions that showed virucidal activity were not considered in determining the reduction in infectivity by the test substance.

Infectivity Assay. The MRC-5 cell line, which exhibits CPE in the presence of Rhinovirus type 37, was used as the indicator cell line in the infectivity assays. Cells in multi-well culture dishes were inoculated in quadruplicate with 0.1 mL of the dilutions prepared from test and control groups. Uninfected indicator cell cultures (cell controls) were inoculated with test medium alone. The cultures were incubated at 31-35° C. in a humidified atmosphere of 5-7% CO₂ in sterile disposable cell culture lab ware. The cultures were microscopically scored periodically for seven days for the absence or presence of CPE, cytotoxicity, and for viability.

Virucidal Results for Test Substance R-400, R-401 and R-402. Results of tests with R-0400, R-0401 and R-0402, exposed to Rhinovirus type 37 in the presence of a 5% fetal bovine serum soil load at 19.5° C. for one minute are discussed below. All cell controls were negative for test virus infectivity. The titer of the dried virus control was 5.5 log₁₀.

Following exposure, test virus infectivity was not detected in the virus-test substance mixture for test substance R-0402 at any dilution tested (≦0.5 log₁₀). Test virus infectivity was detected in the virus-test substance mixture at 2.5 log₁₀ for test substance R-0400 and at 3.75 log₁₀ for test substance R-0401.

Test substance cytotoxicity was observed in test substances R-0400 and R-0402 at 1.5 log₁₀ and in test substance R-0401 at 2.5 log₁₀. The neutralization control (non-virucidal level of the test substance) indicates that the test substances R-0400 and R-0402 were neutralized at ≦1.5 log₁₀ and test substance R-0401 was neutralized at ≦2.5 log₁₀. Taking the cytotoxicity and neutralization control results into consideration, the reduction in viral titer was 3.0 log₁₀ for test substance R-0400, 1.75 log₁₀ for test substance R-0401 and ≧4.0 log₁₀ for test substance R-0402. Accordingly, it was determined that both R-400 and R-401 failed to demonstrate complete inactivation of Rhinovirus type 37. However, results indicated that R-402 demonstrated complete inactivation of Rhinovirus type 37 as required by the U.S. EPA for virucidal label claims. Therefore, the use of R-402 as a virucidal component in a composition is specifically contemplated.

### Example 36

### Virucidal Efficacy of a Disinfectant Against Herpes Simplex Virus type 1 for Use on Inanimate Surfaces

In this study, three different compositions of bentonite clay were studied (R-400, R-401, and R-402) to evaluate their virucidal efficacy against Rhinovirus type 37.

Test Substances.

R-400: purified homoionic sodium bentonite mixture, purified in accordance with U.S. Pat. No. 6,050,509

R-401: purified homoionic hydrogen (protonated) bentonite mixture

R-402: purified homoionic hydrogen (protonated) bentonite #2 mixture

Each of the test substances were dispersed in double distilled water at a concentration of 0.1% (w/v) prior to use in the following assays.

Virus and Preparation of Stock Virus. The F1 strain of Herpes simplex virus type 1 (HSV-1) was obtained from the American Type Culture Collection, Manassas, Va. (ATCC VR-733). Stock virus was prepared by collecting the supernatant culture fluid from infected culture cells. The cells were disrupted and cell debris removed by centrifugation at 2000 RPM for five minutes at approximately 4° C. The supernatant was removed, aliquoted, and the high titer stock virus was stored at ≦−70° C. until the day of use. On the day of use, two aliquots of stock virus (ATS Labs Lot R37-105) were removed, thawed, combined and refrigerated until use in the assay. The stock virus culture contained 1% fetal bovine serum (FBS) as the organic soil load. The stock virus tested demonstrated cytopathic effects (CPE) typical of Herpes simplex virus on rabbit kidney cells.

Test Cell Cultures. Rabbit kidney (RK) cells were obtained from ViroMed Laboratories, Inc., Cell Culture Division. Cultures were maintained and used as monolayers in disposable tissue culture lab ware. On the day of testing, cells were observed as having proper cell integrity.

Test Medium. The test medium used in the following assays was Minimum Essential Medium (MEM), supplemented with 5% (v/v) heat-inactivated fetal bovine serum (FBS), 10 μg/mL gentamicin, 100 U/mL penicillin, and 2.5 μg/mL amphotericin B.

Preparation of Test Substance. Each of R-400, R-401 and R-402 were shaken vigorously by hand for three minutes, aliquoted and utilized immediately in the following assays.

Preparation of Virus Films. Films of virus were prepared by spreading 0.2 mL of virus inoculum uniformly over the bottoms of four separate 100×15 mm sterile glass petri dishes. The virus films were dried at 19.9° C. in a relatively humidity of 70% until visibly dry (approximately 20 minutes).

Sephadex Gel Filtration. To reduce the cytotoxic level of the virus-test substance mixture prior to assay of virus and/or to reduce the virucidal level of the test substance, virus was separated from test substance by filtration through Sephadex gel. Columns of Sephadex LH-20-100 were equilibrated with PBS containing 1% albumin and centrifuged for three minutes to clear the void volume.

Treatment of Virus Films with Test Substance. For each test substance, separate dried virus films were exposed to 2.0 mL of the use dilution for one minute at room temperature (19.9° C.). The virus films were completely covered with the test substance. Following the exposure time, the plates were scraped with a cell scraper to re-suspend the contents of the plate and the virus-test substance mixture was immediately passed through a Sephadex column utilizing a syringe plunger in order to detoxify the mixture. The filtrate (10⁻¹ dilution) was then titered by 10-fold serial dilution and assayed for infectivity.

Treatment of Virus Control Films. A virus film was prepared as described above. The control film was exposed to 2.0 mL of test medium for one minute at room temperature (19.9° C.). The virus was then scraped and passed through a Sephadex column as described above. The filtrate (10⁻¹ dilution) was then titered by 10-fold serial dilution and assayed for infectivity.

Cytotoxicity Assay. A 2.0 mL aliquot of the use dilution of each test substance was filtered through a Sephadex column and the filtrate was diluted serially in medium and inoculated into RK cell cultures. Cytotoxicity of the RK cell cultures was scored at the same time as the virus-test substance and virus control cultures.

Assay on Non-Virucidal Level of Test Substance. Each dilution of the Sephadex-filtered test substance was mixed with an aliquot of low titer stock virus, and the resulting mixtures of dilutions were assayed for infectivity in order to determine the dilution(s) of test substance at which virucidal activity, if any, was retained. Dilutions that showed vinicidal activity were not considered in determining the reduction in infectivity by the test substance.

Infectivity Assay. The RK cell line, which exhibits CPE in the presence of HSV-1, was used as the indicator cell line in the infectivity assays. Cells in multi-well culture dishes were inoculated in quadruplicate with 0.1 mL of the dilutions prepared from test and control groups. Uninfected indicator cell cultures (cell controls) were inoculated with test medium alone. The cultures were incubated at 36-38° C. in a humidified atmosphere of 5-7% CO₂ in sterile disposable cell culture lab ware. The cultures were microscopically scored periodically for seven days for the absence or presence of CPE, cytotoxicity, and for viability.

Virucidal Results for Test Substance R-400, R-401 and R-402. Results of tests with R-0400, R-0401 and R-0402, exposed to HSV-1 in the presence of a 5% fetal bovine serum soil load at 19.9° C. for one minute are discussed below. All cell controls were negative for test virus infectivity. The titer of the dried virus control was 6.25 log₁₀.

Following exposure, test virus infectivity was not detected in the virus-test substance mixture for test substance R-0402 at any dilution tested (≦0.5 log₁₀). Test virus infectivity was detected in the virus-test substance mixture at 3.5 log₁₀ for test substance R-0400 and at 3.25 log₁₀ for test substance R-0401.

Test substance cytotoxicity was not observed in test substances R-0400 and R-0402 at any dilution tested (≦1.5 log₁₀). Test substance cytotoxicity was observed in R-401 at 2.5 log₁₀. The neutralization control (non-virucidal level of the test substance) indicates that the test substances R-0400 and R-0402 were neutralized at ≦0.5 log₁₀ and test substance R-0401 was neutralized at ≦2.5 log₁₀. Taking the cytotoxicity and neutralization control results into consideration, the reduction in viral titer was 2.75 log₁₀ for test substance R-0400, 3.0 log₁₀ for test substance R-0401 and ≧5.75 log₁₀ for test substance R-0402. Accordingly, it was determined that both R-400 and R-401 failed to demonstrate complete inactivation of Rhinovirus type 37. However, results indicated that R-402 demonstrated complete inactivation of Rhinovirus type 37 as required by the U.S. EPA for virucidal label claims. Therefore, this example further supports the use of R-402 as a virucidal component in a composition.

1. A method of inhibiting transfer of a virus to a surface, the method comprising contacting the surface with a composition comprising a layered phyllosilicate material in an amount sufficient for inhibiting the transfer of the virus thereto.

2. The method of claim 1, wherein the virus is selected from the group consisting of Feline Calcivirus, Norovirus, Rotavirus, HSV-1, Influenza A, HIV and Rhinovirus.

3. A method in accordance with claim 1, wherein the layered phyllosilicate material has at least 90% homoionic interlayer exchangeable cations, in relation to all interlayer exchangeable cations, and has a particle size less than 74 μm.

4. The method of claim 3, wherein the phyllosilicate material has interlayer exchangeable cations that are predominantly hydrogen cations.

5. A method in accordance with claim 1, wherein the layered phyllosilicate material comprises exfoliated platelets and/or tactoids of the layered phyllosilicate material.

6. The method of claim 1, wherein the composition is in a form selected from the group consisting of a solution, lotion, cream, ointment, powder, suspension, stick, gel, aerosol, or nonaerosol pump spray.

7. The method of claim 1, wherein the surface is an inanimate surface.

8. The method of claim 1, wherein the surface is an animate surface.

9. The method of claim 8, wherein the animate surface is on a mammalian subject.

10. The method of claim 9, wherein the mammalian subject is selected from the group consisting of a human, a horse, a dog, a cat; a dromedary, an exotic animal, a zoo animal, a mouse, a rat, a rabbit, a guinea pig, and a hamster.

11. The method of claim 9, wherein the animate surface is human skin.

12. A method of inactivating a virus on an animate surface comprising contacting said surface with a composition comprising a layered phyllosilicate material in an amount sufficient to inactivate said virus.

13. The method of claim 12, wherein the virus is selected from the group consisting of Feline Calcivirus, Norovirus, Rotavirus, HSV-1, Influenza A, HIV and Rhinovirus.

14. The method of claim 12, wherein the layered phyllosilicate material has at least 90% homoionic interlayer exchangeable cations, in relation to all interlayer exchangeable cations, and has a particle size less than 74 μm.

15. The method of claim 12, wherein the phyllosilicate material has interlayer exchangeable cations that are predominantly hydrogen cations.

16. A method in accordance with claim 12, wherein the layered phyllosilicate material comprises exfoliated platelets and/or tactoids of the layered phyllosilicate material.

17. The method of claim 12, wherein the composition is in a form selected from the group consisting of a solution, lotion, cream, ointment, powder, suspension, stick, gel, aerosol, or nonaerosol pump spray.

18. The method of claim 14, wherein the animate surface is on a mammalian subject.

19. The method of claim 18, wherein the mammalian subject is selected from the group consisting of a human, a horse, a cow, sheep, a pig, a llama, an alpaca, a goat, a dog, a cat; a dromedary, an exotic animal, a zoo animal, a mouse, a rat, a rabbit, a guinea pig, and a hamster.

20. The method of claim 18, wherein the animate surface is human skin.

21. A method of inactivating a virus on an inanimate surface comprising contacting said surface with a composition comprising a layered phyllosilicate material in an amount sufficient to inactivate said virus.

22. The method of claim 21, wherein the virus is selected from the group consisting of Feline Calcivirus, Norovirus, Rotavirus, HSV-1, Influenza A, HIV and Rhinovirus.

23. The method of claim 21, wherein the layered phyllosilicate material has at least 90% homoionic interlayer exchangeable cations, in relation to all interlayer exchangeable cations, and has a particle size less than 74 μm.

24. The method of claim 21, wherein the phyllosilicate material has interlayer exchangeable cations that are predominantly hydrogen cations.

25. The method of claim 21, wherein the layered phyllosilicate material comprises exfoliated platelets and/or tactoids of the layered phyllosilicate material.

26. The method of claim 21, wherein the composition is in a form selected from the group consisting of a solution, lotion, cream, ointment, powder, suspension, stick, gel, aerosol, or nonaerosol pump spray.
